(19) United States
(12) Patent Application Publication
(10) Pub. No.: US 2025/0064571 A1
(43) Pub. Date: Feb. 27, 2025

(54) TISSUE MATRIX PACKAGING SYSTEM

(71) Applicant: Musculoskeletal Transplant Foundation, Edison, NJ (US)

(72) Inventors: Jeffrey Kapec, Westport, CT (US); Yukiko Naoi, New York, NY (US); Alison Jill Ling, Piscataway, NJ (US); Scott Tisi Lansdale, Bordentown, NJ (US); Dirrin Nathan Friend, Robbinsville, NJ (US)

(21) Appl. No.: 18/842,279

(22) PCT Filed: Mar. 3, 2023
(86) PCT No.: PCT/US2023/014493

§ 371 Date: Aug. 28, 2024

Related U.S. Application Data
(60) Provisional application No. 63/316,368, filed on Mar. 3, 2022.

(51) Int. Cl. A61F 2/00; A61L 27/36

(52) U.S. Cl. CPC A61F 2/0095; A61L 27/3691; A61L 2430/40

(57) ABSTRACT

A multi-component packaging system capable of holding a graft and fluid therein, and which enables separation and removal of fluid from the graft, hydration of the graft by adding fluid, or both, while the graft is held in the packaging system. The packaging system comprises: a graft holding region for holding the graft and, optionally, a fluid; a reservoir region for receiving and containing fluid separated from the graft; a container configured to hold the graft, prevent contamination, and which defines the reservoir region. The packaging system further comprises: at least one fluid separation feature for enabling separation of fluid from the graft; at least one compression feature for applying compressive force to assist with more efficient separation of fluid from the graft and displacement of separated fluid from the graft holding region and into the reservoir region, and at least one fluid communication means which allows displaced fluid to leave the graft holding region and enter the reservoir region.

### CROSS-REFERENCE TO RELATED APPLICATIONS

The present application is a national stage of International Patent Application No. PCT/US2023/014493, filed Mar. 3, 2023, which claims priority from U.S. Provisional Application No. 63/316,368, filed Mar. 3, 2022, the entire disclosures of both of which are incorporated by reference herein.

### FIELD OF THE INVENTION

The present invention relates to packaging systems for containing, transporting, storing, and producing grafts used for medical treatments.

### BACKGROUND

Packaging for containing grafts useful for medical treatments are generally designed and configured to maintain the condition of the grafts from the time of their production, to storage, transport and use. Packaging is designed to maintain one or more graft conditions including, but not limited to, shape, configuration, moisture content, degree of disinfection or sterilization, viability, bioactivity, as well as many others.

Furthermore, instead of waiting until the end of production or manufacture to place a finished graft in the packaging, some graft packaging includes features which enable or facilitate performing one or more of the production processes while the graft is contained in the packaging. Some such production processes include, but are not limited to, contacting or soaking with preservative solution, rinsing, soaking, freezing, staging (e.g., interim storage or holding periods), cryopreserving, lyophilizing, dehydrating, disinfecting, sterilizing, and many others.

Grafts provided in packaging may have any one or more of several possible conditions and characteristics such as, without limitation, hydrated, dehydrated, partially dehydrated, in contact with or immersed in a fluid or other material, in a frozen state, or any of many other possible conditions and properties. Depending on the condition and characteristics of a packaged graft and the desired use of the graft, it may be beneficial or even necessary to perform one or more preparation processes to modify one or more conditions and characteristics of the graft before it is used for medical treatments. Such preparation processes include, without limitation, thawing, rinsing, contacting or combining the graft with a fluid or liquid, separation and removal of excess fluid or liquid, adding tissue, adding substances, and other processes which modify or enhance desired handling and performance characteristics of the graft.

While graft packaging may include a single component, multi-component packaging systems have become prevalent because they often provide reliable and consistent protection and maintenance of graft conditions, as well as enabling the performance of one or more production and preparation processes. More particularly, some packaging systems include two, three, or even more components, which fit or otherwise cooperate together to provide the desired degree of protection and stability of the graft during at least one of production, transport, storage, and use of the graft.

In the foregoing circumstances, and given constantly advancing developments in graft technology, there is an ongoing need for packaging having new and improved features and capabilities which provide the ability to selectively maintain and modify conditions and characteristics of grafts contained in the packaging.

### SUMMARY

A multi-component packaging system is provided for holding a graft therein during hydrating of the graft, separating excess fluid from a hydrated graft, or both. More particularly, the multi-component packaging system includes: a graft holding region for holding the graft and, optionally, a fluid; a reservoir region for receiving and containing fluid; a container having one or more walls and a bottom which define the reservoir region, and a top portion having an open top, which allows fluid to be provided to the reservoir region, and an inner top cross-sectional area; and one or more fluid communication means which provides fluid communication between the graft holding region and the reservoir region. The graft holding region and reservoir region may be at least partially coextensive.

The multi-component packaging system further includes: one or more fluid separation features for separating fluid from a graft held in the graft holding region; and one or more compression features for applying compressive force to a graft while held in the graft holding region and causing displacement of fluid from the graft, from the graft holding region, through the one or more fluid communication means, and into the reservoir region. The one or more fluid separation features and the one or more compression features may be at least partially the same structure or not.

A method for separating and displacing fluid from a graft using the aforesaid multi-component packaging system is also provided. More particularly, the method comprises: assembling the multi-component packaging system with a graft held in the graft holding region, displacing fluid from the graft by applying compressive force to the graft using the one or more compression features, then allowing displaced fluid to move from the graft holding region, through the fluid communication means, to the reservoir region, before separating the graft from the reservoir region, and separating the graft from the reservoir region and displaced fluid therein, if any, using the one or more fluid separation features.

The method may further comprise, prior to displacing fluid from the graft, further hydrating or rehydrating the graft by adding fluid to the reservoir region and pausing for a time sufficient to hydrate the graft to a desired degree. The method may further comprise, prior to or during allowing displaced fluid to move from the graft holding region, driving displaced fluid from the graft holding region, through the fluid communication means, to the reservoir region, using the one or more compression features.

In a first exemplary embodiment of the multi-component packaging system, the one or more fluid separation features comprises: a first straining basket with an open top, a bottom, a wall, a first inner cross-sectional area or diameter of uniform size and a first outer cross-sectional area or diameter of uniform size; and a second straining basket with an open top, a bottom, a wall, a second inner cross-sectional area or diameter of uniform size, and a second outer cross-sectional area or diameter of uniform size.

In embodiments of the multi-component packaging system which include first and second straining baskets, the first outer cross-sectional area or diameter of the first straining basket is sized and shaped to allow the first straining basket to slidingly fit through the inner top cross-sectional area of the container and into the container, the first straining basket occupying at least a portion of the reservoir region of the container. Furthermore, the second outer cross-sectional area or diameter of the second straining basket is sized and shaped to allow the second straining basket to slidingly fit through the first inner cross-sectional area of the first straining basket and into the first straining basket, and the graft holding region is defined by: the bottom of the first straining basket, the bottom of the second straining basket, and at least a portion of each of the one or more walls of the container, the graft holding region occupying at least a portion of the reservoir region

In some embodiments of the multi-component packaging system which include first and second straining baskets, the one or more fluid communication means may comprise at least a first fluid communication means which comprises a mesh material, a plurality of apertures, or both, and is provided either: on at least a portion of the bottom of the first straining basket, or on at least a portion of the bottom of the second straining basket.

In other embodiments of the multi-component packaging system which include first and second straining baskets, the one or more fluid communication means may comprise at least a first fluid communication means and a second fluid communication means, each of which comprises a mesh material, a plurality of apertures, or both, and the first straining basket includes the first fluid communication means which is provided on at least a portion of the bottom of the first straining basket, and the second straining basket includes the second fluid communication means which is provided on at least a portion of the bottom of the second straining basket.

In some embodiments of the multi-component packaging system which include first and second straining baskets, the one or more compression features may comprise at least a primary compression feature which includes the bottom of the second straining basket. When the second straining basket is slidingly inserted into the first straining basket, the bottom of the second straining basket is capable of contacting and applying compressive force against a graft held in the graft holding region, and the second straining basket applies compressive force on the graft, fluid is separated and displaced from the graft, and the first communication means and the second communication means allow separated and displaced fluid to leave the graft holding region and enter the reservoir region of the container.

In a second exemplary embodiment of the multi-component packaging system, the container has a uniform inner cross-sectional area which is equal to inner top cross-sectional area and extends from the top portion to the bottom of the container, and the one or more fluid separation features may comprise a first plunger, or a first plunger and a second plunger, each of which includes a compression plate and a vertically-oriented element extending therefrom.

In embodiments of the multi-component packaging system wherein the one or more fluid separation features comprises only a first plunger, the first plunger includes: a first compression plate which is sized and shaped to fit slidingly into the container and extends across the uniform cross-sectional area of the container, and a first vertically-oriented element which extends upward from the compression plate and facilitates moving the first plunger slidingly in the container and removing the first plunger from the container, and the one or more fluid communication means comprise at least a first fluid communication means which comprises a mesh material, a plurality of apertures, or both, and is provided on at least a portion of the first compression plate.

In embodiments of the multi-component packaging system wherein the one or more fluid separation features comprises only a first plunger, the first compression plate of the first plunger, the bottom of the container, and at least a portion of each of the one or more walls of the container, together, define the graft holding region, the graft holding region occupying at least a portion of the reservoir region of the container. Furthermore, the one or more compression features comprise at least the first compression plate of the first plunger and, when the first plunger is slidingly inserted into container, the first compression plate is capable of contacting and applying compressive force against a graft held in the graft holding region, and when the first compression plate applies compressive force on the graft, fluid is separated and displaced from the graft, and the first communication means allows separated and displaced fluid to leave the graft holding region and enter the reservoir region of the container.

In embodiments of the multi-component packaging system wherein the one or more fluid separation features comprises the aforesaid first plunger as well as a second plunger, second plunger which includes: a second compression plate which is sized and shaped to correspond to the first compression plate of the first plunger and which fits slidingly into the container and extends across the uniform cross-sectional area of the container, and a second vertically-oriented element extending upward from the second compression plate, wherein the one or more compression features further comprises the second compression plate of the second plunger. Furthermore, the first vertically-oriented element of the first plunger includes a vertically-oriented opening therethrough and the second vertically oriented element is positioned on the second compression plate in alignment with the vertically-oriented opening and is sized and shaped to be slidingly received through the vertically-oriented opening when the packaging system is assembled.

In embodiments of the multi-component packaging system wherein the one or more fluid separation features comprises the aforesaid first plunger as well as a second plunger, the packaging system is assembled having the second compression plate of the second plunger slidingly inserted in the container, proximate to or contacting the bottom of the container, and having the first compression plate of the first plunger slidingly inserted in the container, above the second compression plate, whereupon the first compression plate, the second compression plate, and at least a portion of each of the one or more walls of the container, together, define the graft holding region, and the graft holding region occupies at least a portion of the reservoir region of the container.

In its assembled configuration, embodiments of the multi-component packaging system comprising aforesaid first and second plungers, the one or more compression features comprise at least the first compression plate of the first plunger which is capable of contacting and applying compressive force against a graft held in the graft holding region. Furthermore, when the first compression plate applies compressive force on the graft, fluid is separated and displaced from the graft, and the first communication means provided on at least a portion of the first compression plate allows separated and displaced fluid to leave the graft holding region and enter the reservoir region of the container.

In its assembled configuration, embodiments of the multi-component packaging system comprising aforesaid first and second plungers have the second vertically-oriented element of the second plunger is assembled with and slidingly received in the vertically-oriented opening of the first vertically-oriented element of the first plunger, and the assembled first and second vertically-oriented elements are useful as a handle for concurrently removing the first plunger and second plunger from the container.

### BRIEF DESCRIPTION OF THE FIGURES

The present invention will be further explained with reference to the attached drawings, wherein like structures are referred to by like numerals and/or letters throughout the several views. The drawings shown are not necessarily to scale, with emphasis instead generally being placed upon illustrating the principles of the present invention.

FIG. 1 is a front elevational cut-away view of an exemplary embodiment of a first component of packaging in accordance with the present disclosure, in an assembled state and showing how its elements fit together;

FIG. 2 is a partially exploded top perspective view of the first component of FIG. 1, showing its elements;

FIG. 3 is an exploded bottom perspective view of the first component of FIG. 1, showing its elements;

FIG. 4 is an exploded top perspective view the first component of FIG. 1, showing its elements;

FIG. 5 is a front elevational cut-away view of a modified first component similar to that of FIG. 1, but having differently sized mesh;

FIG. 6 is a front elevational cut-away view of another modified first component similar to that of FIG. 1, but having floor elements with apertures rather than mesh floors;

FIG. 7 is another front elevational cut-away view of the modified first component of FIG. 6;

FIG. 8 is an exploded top perspective view the modified first component of FIGS. 6 and 7, showing its elements;

FIG. 9 is an exploded bottom perspective view of the modified first component of FIGS. 6 and 7, showing its elements;

FIG. 10 is an exploded front perspective view of another exemplary embodiment of a first component of packaging in accordance with the present disclosure, showing its elements as well as a properly positioned graft;

FIG. 11 is an exploded top perspective view of the first component of FIG. 10, showing the arrangement of its elements and a graft;

FIG. 12 is an exploded front perspective view of the first component of FIG. 10, in its partially assembled state, with a graft and before the lid has been closed;

FIG. 13 is an exploded top perspective view of modified first component similar to that of FIGS. 10-12, but lacking a second plunger;

FIG. 14 is a front perspective view of another modified first component having elements similar to that of FIGS. 10-12, but being made of translucent material and in its partially assembled state, with a graft positioned therein;

FIG. 15 is an exploded top perspective view of the modified first component of FIG. 14, showing its elements and their arrangement;

FIG. 16 is an exploded top perspective view of the modified first component of FIG. 14, showing how the first and second plungers fit together;

FIG. 17 is an exploded top perspective view of the modified first component of FIG. 14, showing the arrangement of elements and a graft mounted on the second plunger;

FIG. 18 is an exploded top perspective view of the modified first component of FIG. 17, in its partially assembled state, with the second plunger and graft mounted thereon positioned in the container element;

FIG. 19 is a top perspective view of the modified first component of FIG. 19, in its assembled state with a graft positioned between the first and second plungers and in the reservoir region of the container element and a lid sealingly fitted on the container;

FIG. 20 is a perspective view of another exemplary embodiment of a first component of packaging in accordance with the present disclosure, showing its elements which include a container and a mesh pouch;

FIGS. 21 and 22 both provide perspective views of the mesh pouch shown in FIG. 20 and a compression element clamped on an upper portion of the mesh pouch element, where FIG. 21 shows the compression element at or near the beginning of its path and FIG. 22 shows the compression element at or near the end of its path;

FIG. 23 is an exploded top perspective view of another exemplary embodiment of a first component of packaging in accordance with the present disclosure, showing its elements which include a container, a lid, and a modified compression element for operation with a mesh pouch (not shown);

FIG. 24 is an exploded front elevational view of the first component of FIG. 23;

FIG. 25 is an exploded side elevational view of the first component of FIG. 23;

FIG. 26 is an exploded top perspective view of the first component of FIG. 23, showing its elements (now including a mesh pouch) and their arrangement;

FIG. 27 is an exploded front elevational view of the first component of FIG. 26;

FIG. 28 is a partially exploded top perspective view of the first component of FIG. 26, showing the mesh pouch and a modified compression element assembled together for operation;

FIG. 29 is a front elevational view of the first component of FIG. 28;

FIG. 30 is a side elevational view of the first component of FIG. 28;

FIG. 31 is a perspective view of another exemplary embodiment of a first component of packaging, in accordance with the present disclosure, in its assembled state; and

FIG. 32 is a perspective view of the first component of FIG. 31 in its disassembled state.

### DETAILED DESCRIPTION

Generally, unlike existing packaging for grafts which contain, or are combined or have been rehydrated, with a biocompatible fluid and for which simple gravity separation or decanting of the fluid from the graft is too slow or difficult to be practicable at the point of use, the graft packaging contemplated and described in detail below includes both a fluid separation feature which allows separation of fluid from a graft while positioned within the packaging, and also a compression feature for applying compressive force to the graft and causing separation and displacement of fluid from the graft, thereby increasing the quantity and efficiency of separating and removing fluid from a graft while held in the packaging.

Grafts suitable for use in medical treatments and procedures such as, without limitation, for wound healing or tissue repair and reconstruction, whether due to trauma, disease or cosmetic modification, are made from any of a vast number of different materials. Suitable materials for making grafts are biocompatible and often also possess other beneficial characteristics and properties which enable or enhance structural properties or biological properties of the graft, or both. Grafts of all sizes, shapes and configurations have been successfully produced from metals, alloys, fabrics, meshes, polymers, resins, natural materials, synthetic materials, and combinations thereof. Tissue derived matrices are a naturally sourced material used, alone or in combination with other suitable materials, to make grafts which are diverse and widely useful for all sorts medical treatments and procedures. Tissue derived matrices may be produced from autogenic, allogenic or xenogenic tissue samples. Furthermore, the tissue samples may be any of several tissue types including, but not limited to: bone (cortical, cancellous), cartilage, dermis, adipose, placenta, fascia, and combinations thereof.

While packaging in accordance with the invention described and contemplated herein is particularly suitable for and described in connection with grafts comprising tissue derived matrices and which are at least somewhat compressible and may be provided in a hydrated state (i.e., not dehydrated or already combined or in contact with a fluid), or initially dehydrated state and then rehydrated or otherwise combined or in contact with a fluid, the packaging is equally applicable and useful for use with grafts made from other materials and combinations thereof. For instance, any grafts containing or combined with a fluid and which are required, or for which it would be beneficial, to be separated from the fluid prior to use, would benefit from use or combination with the packaging described and contemplated herein which enables or facilitates such separation while still contained in packaging. The packaging described and contemplated herein is applicable and useful with grafts which are at least somewhat compressible, regardless of the material from which they are made or the physical form in which they are provided (i.e., particles, pieces, fibers, sheets, geometric or irregular three dimensional shapes, etc.).

Depending on the configuration and nature of the materials of which a graft is made, the graft may be produced and packaged in a hydrated or dehydrated state, with or without fluid in contact with the graft, in a frozen state, or in any of many other possible conditions and properties. Depending on the condition and properties of the graft contained in packaging, some grafts need to be thawed, hydrated, dehydrated, separated from solution or media present in the packaging with the graft. or subjected to one or more other preparation processes, prior to actual use. Packaging has and is being developed which enables or enhances the ability to efficiently and successfully perform one or more such preparation processes while the graft remains in the packaging.

“Biocompatible fluid” means a fluid which will not produce a toxic, injurious, or immunologic response when contacted with living tissue. Examples of biologically compatible fluids suitable for combination with one or more protectants include, without limitation: an aqueous buffer (for example without limitation, phosphate buffered saline (“PBS”)), a buffered or non-buffered isotonic solution (for example without limitation, an aqueous sodium chloride solution (e.g., from about 0.1 weight % (wt %) to about 1 wt %, such as about 0.9 wt %), a lactated Ringer's solution, a cell storage or culture media (for example without limitation, DMEM, a basal medium (e.g., Basal Medium Eagle (BME), or other similar media)), platelet rich plasma (PRP), lecithin, alginate, hyaluronic acid (HA), a derivative or salt of HA (e.g., sodium hyaluronate), bone marrow, other suitable biologically compatible fluids known to persons of ordinary skill in the relevant art, and mixtures thereof.

Unless otherwise specified, the term “fluid” includes any gas, vapor, liquid, particulate solids, and any combination, mixture, solution, slurry, or suspension, thereof, which is flowable and capable of being provided to and received by packaging or by one or more components of packaging.

“Hydrate” and “rehydrate” and all of their grammatical forms, as used herein, refer to the process of contacting or otherwise combining a graft with one or more biocompatible fluids, upon which the graft absorbs or otherwise mixes with the fluid which typically modifies handling characteristics and other properties of the graft. For example, a hydrated or rehydrated graft is expected to be more flexible, pliable, and even moldable and reshapable, while becoming less stiff and brittle as compared to prior to hydration or rehydration. In some embodiments, such as where the grafts comprise one or more tissue derived matrices which contain cells, rehydrating with a biologically compatible fluid, may reinvigorate dormant cells, e.g., bring cells from a dormant state to a state in which evidence of metabolic activity can be detected. Where a preserved tissue has been frozen, the preserved tissue may be thawed before, during, or after contacting with a biologically compatible fluid.

As used herein, “packaging” means and includes any device which at least partially contains or retains a graft thereon or therein, whether in an opening, cavity, recess, or layered with or proximate to one or more components of the packaging device, and provides one or more benefits or protections for the graft.

Grafts useful for medical treatments are often placed in packaging for storage and transport. Such packaging is designed and configured to contain the grafts, as well as to maintain conditions and characteristics of the grafts, during their storage and transportation. Such graft conditions and characteristics include, but are not limited to, shape, configuration, moisture content, degree of disinfection or sterilization, viability, bioactivity, and many others.

Rather than completing production of a graft and then placing it in packaging, it may be advantageous to perform one or more production processes while the graft is contained in the packaging in which it will later be transported, stored, or both. Such production processes include, without limitation, freezing, dehydrating, sterilizing, and other processes which contribute to production of the graft.

For instance, after a graft is frozen it may be difficult to transfer the frozen graft from a container or vessel in which it was frozen to packaging suitable for transport and storage of the frozen graft. Additionally, it may be more efficient to cryopreserve or lyophilize a graft while it is in packaging, which typically involves contacting or combining the graft with a suitable preservation solution, and/or other substances, by adding the solution to the packaging, and then either cryopreserving or lyophilizing the graft and preservation solution together in the packaging. After cryopreserving, the preservation solution (and/or other added fluids) may be separated and removed from the graft and packaging, or not. In fact, there are several processes during production of grafts which may involve combining or contacting the graft with one or more substances and materials, for any of several reasons and purposes, and possibly removing such substances and materials later, any and all of which are contemplated herein as possibly being performed while the graft is disposed in packaging.

As another example, once a graft has been disinfected or sterilized, transferring the disinfected or sterilized graft from a container, vessel, or environment in which it was disinfected or sterilized may provide an opportunity or even likelihood of subsequent contamination of the graft, which would require repeating disinfection or sterilization. Thus, disinfecting a graft, sterilizing a graft, or both, while the graft is contained in packaging is likely more effective and efficient.

Furthermore, it is often advantageous, or even necessary, to modify one or more conditions and characteristics of a packaged graft prior to its use. Such preparation processes include, but are not limited to: thawing, rinsing, contacting or combining the graft with a fluid or liquid, separation and removal of excess fluid or liquid, adding substances, and other processes which modify conditions and characteristics of the graft. Such modifications often enhance desired handling properties and performance characteristics of the graft, which may facilitate placement and effectiveness of the graft. Oftentimes it is more efficient and effective to perform preparation processes which modify the graft as desired while the graft is still in the packaging and then remove the graft for use.

Grafts comprising one or more tissue derived matrices and which are at least somewhat compressible, regardless of the physical form in which they are provided (i.e., particles, pieces, fibers, sheets, geometric or irregular three dimensional shapes, etc.) may be provided in packaging in a dehydrated state, in which case rehydrating with one or more biocompatible fluids is typically performed, or in a hydrated or previously rehydrated state. Prior to use, it is sometimes advantageous to separate and remove at least a portion of the fluid from such packaged graft prior to use, to modify one or more handling characteristics and properties.

In particular, the nature of bone fiber allograft matrix is such that it readily absorbs fluids and swells as it absorbs fluids. This may produce an oversaturated graft, when rehydrated at the point of use, that is too wet and not cohesive enough to form the desired moldable putty useful for treatment of bone void defects. Such situations result in the need for practitioners using the graft to be very careful about the quantity of fluid used to rehydrate bone fiber grafts or somehow separate and remove at least a portion of the fluid after it is determined that the handling characteristics of the graft require modification for the intended use. The presently described and contemplated invention provides packaging with a compression feature which enables easier separation and removal of liquid than trying to strain, wring and/or squeeze an oversaturated graft.

Conventional packaging and packaging systems include features such as filters, meshes, and sieves which have been used as fluid delivery features for delivering one or more biocompatible fluids to hydrate or rehydrate the graft while positioned in the packaging. If used for fluid separation, these features must operate and rely on gravity or other passive physical mechanisms to achieve separation of fluid from the graft.

The packaging in accordance with the invention described and contemplated herein include features to facilitate rehydration (i.e., delivery of biocompatible fluid to contact a graft contained in the packaging, as well as homogenous distribution or contact of the fluid with the graft), as well as separation and removal of excess rehydration fluid (and other fluid, if present) with minimal steps to achieve desired handling properties in preparation for implantation/surgical use. Whether there is “excess” fluid in or with the graft will be determined at the time of use depending upon the circumstances and intended use at the time of rehydration and use.

As will be discussed in further detail below, compression features of the packaging described and contemplated herein enable application of compressive force to a graft inside the packaging, thereby facilitating and accelerating separation of fluid from the graft. Several embodiments of such packaging and will now be described.

Generally, packaging systems for containing, storing and transporting grafts comprise two, three, or more components, which fit or otherwise cooperate together to provide the necessary degree of protection, stability, and manipulation of the graft during one of more of production, transport, storage, and use of the graft. Overall, the packaging system should provide protection of the graft according to industry expectation including, without limitation, preservation of sterility, cell viability, moisture content, physical protection against damage during shipping and handling and over the shelf-life of the graft.

In some embodiments, a multi-component packaging system comprises at least a first component, e.g., a primary container, which is configured to hold a graft such that it prevents contamination while enabling lyophilization of the graft. Accordingly, the primary container should include venting features to adequately allow removal and escape, during lyophilization, of inherent moisture from the graft, as well as fluids that may have been added during the manufacturing process. Additionally, the primary container may preferably be constructed of materials that prevent or minimize moisture ingress to the graft after lyophilization.

Generally, the packaging system may further comprise a second component, e.g., a secondary outer package, which is sized and shaped to receive and retain the primary container with the graft held therein. The secondary outer package may, for example, be designed to prevent contamination of the graft as well as provide moisture control (allowing or preventing moisture vapor ingress or contacting fluid with the graft). In some embodiments, the secondary outer package may be a thermoformed or injection molded plastic tray with a peelable cover or lid to enclose the tray and retain the primary container therein. The secondary outer tray may be produced from standard medical device materials, such as are known to persons of ordinary skill in the relevant art.

The packaging system may further comprise a third component, e.g., a tertiary carton, pouch, or other container for shipping. The tertiary carton is sized and shaped to receive and contain the secondary outer package therein package. The tertiary carton may, for example, be configured and designed to provide additional cushioning against physical damage during transport, as well as separation and protection of the graft from ambient conditions and printed or otherwise affixed information in its outer surface relating to identification, description, expiration, intended use, manufacturer, etc., of the graft contained in the packaging system.

Packaging for grafts described and contemplated herein comprises at least a first component (such as the primary container mentioned above) which includes: a graft holding region for holding the graft and, optionally, a fluid; and a reservoir region for receiving and containing fluid separated from the graft. The graft holding region of the first component is sized and shaped to provide a capacity which is able to contain a predetermined volume of fluid. It is contemplated that, in some embodiments the graft holding region and the reservoir region may be at least partially coextensive or overlapping (i.e., at least partially the same) with one another, or one may fit at least partially within the other. The first component further includes at least one first opening which provides fluid communication between the graft holding region and the reservoir region to enable providing fluid during rehydration, or separating and removing fluid when it is determined that excess fluid is present in the graft or with the graft in the graft holding region.

The first component also includes at least one compression feature for applying compressive force to a graft positioned in the graft holding region and displacing fluid from the graft holding region. A second opening in the first component is also provided and in fluid communication between the reservoir region and an environment which is external to the packaging.

In some embodiments, the packaging may include a removable cover or lid (such as, without limitation, a peelable planar lid) for sealingly closing the second opening (i.e., to prevent or minimize unwanted escape of fluid and/or graft material from the packaging. For example, without limitation, the first component may include a press-fit plastic closure, a screw-top plastic cap, rubber stopper, or peelable foil lid with a vent (such as a porous cap liner) which may be adhered, for example, using heat sealing or induction sealing, breathable plastic membrane or mesh, Tyvek, or a physical opening in the closure. In some embodiments, the cover or lid may be include locking means to prevent unintended opening of the container which might prematurely or otherwise undesirably expose the graft and/or fluid. In some embodiments, one or both of the packaging and cover or lid may be provided with space for addition of labeling or symbols.

In some embodiments, the first component (i.e., a primary container) is manufactured with a transparent or translucent moisture barrier which is capable of preventing moisture ingress or preventing moisture loss at a rate of less than or equal to 20%. Suitable materials include, but are not limited to, cyclic olefin polymers and copolymers (COP, COC), which may be injection molded or subjected to another suitable manufacturing technique applicable to such polymer materials to achieve a moisture vapor transmission rate of from about 0.1 to about 2.0 mg/cavity/day. Thermoformed heat sealable film is another suitable type of moisture barrier for construction of the primary container or a portion thereof, and could be made materials such as, but not limited to polychlorotrifluoroethylene (PCTFE), polyester (PET), polyethylene (PE), polyethylene terephthalate glycol (PETG), and combinations thereof. It is beneficial is the materials used for construction of the first component (primary container) are also compatible and suitable for typical sterilization methods such as autoclaving, irradiation, etc.

In some embodiments, the packaging may further include: a sealable inlet in fluid communication with the graft holding region for supplying a fluid to the packaging and allow the fluid to contact the graft therein; and a sealable outlet in fluid communication with the enclosed region for allowing fluid separated from the graft and displaced from the graft holding region to the enclosed region to flow or be displaced from the enclosed region and packaging.

A method of separating and removing fluid from a graft positioned in a graft holding region of the above-described first component (i.e., also including a reservoir region, a compression feature; at least one first opening; and a second opening in a closed state), regardless of whether the fluid is present in the graft or with the graft in the graft holding region, or was added to the graft holding region at some time after the graft was positioned therein, may be performed by applying compressive force to the graft using the compression feature to separate fluid from the graft and displace separated fluid from the graft holding region through the at least one first opening and into the reservoir region.

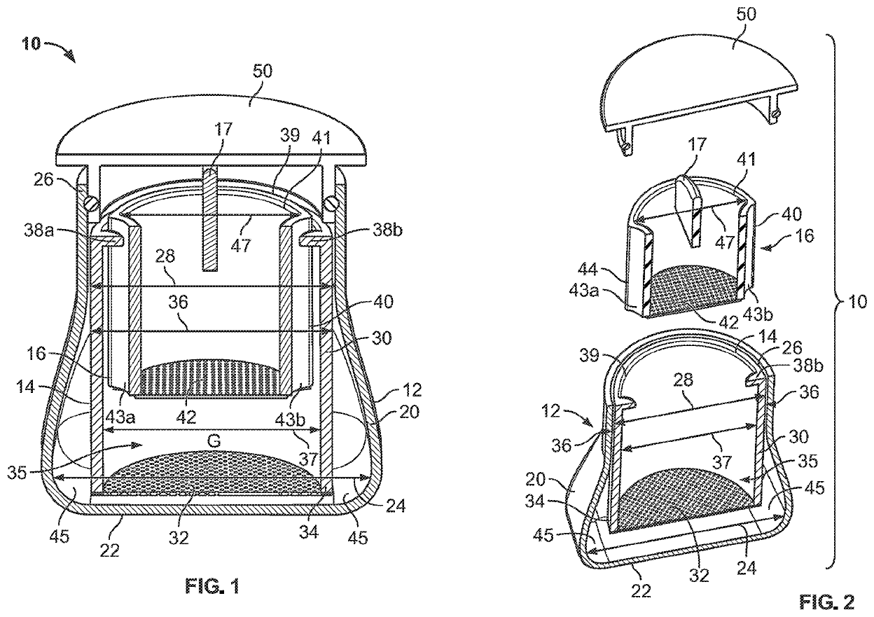

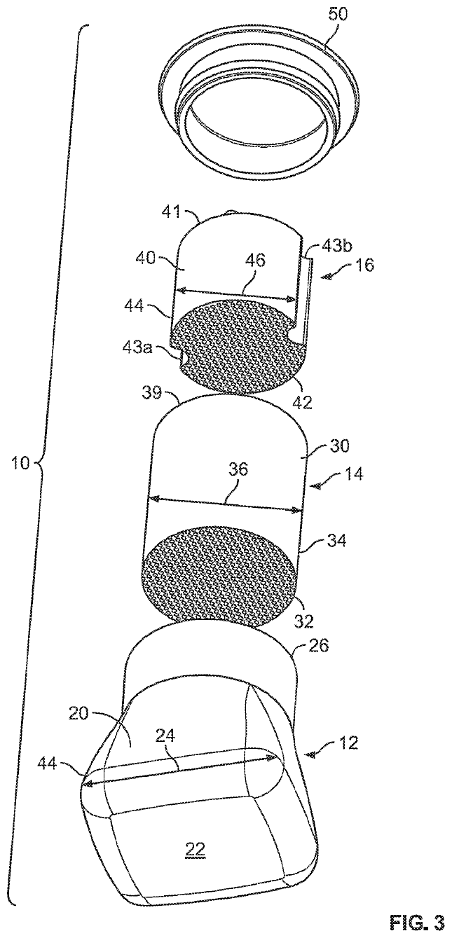

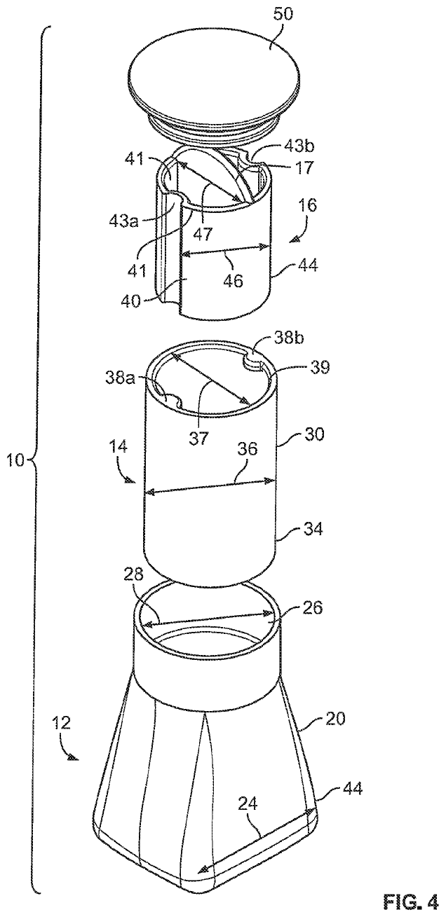

With reference now to FIGS. 1-4, a first embodiment of a first component 10 of a multi-component packaging system is shown in an assembled state with a cut-away front view to show how its elements fit together. FIG. 2 provides a partially exploded view, and each of FIGS. 3 and 4 provide exploded views, of the first component 10 and its elements which include a rigid outer container 12, a first nesting straining basket 14 which is generally sized and shaped to fit within the rigid outer container 12, and a second nesting straining basket 16 which is generally sized and shaped to nestingly fit within the first nesting straining basket 14. Other components of the packaging system are not shown, but are of conventional construction (i.e., a second component being sized and shaped to contain the first component 10 therein, and a third component being sized and shaped to contain the second component therein, and both having the characteristics discussed above).

In the embodiment shown in FIGS. 1-4, the rigid outer container 12 includes a vertically oriented container wall 20, which joins a solid container bottom 22 and has a first inner cross-section 24 proximate the container bottom 22. In some embodiments such as that shown in FIGS. 1-2, the bottom shape of the container 12 may be a curvilinear shape. The wall 20 of the outer container 12 transitions and tapers to form a cylindrical open neck top 26 (see FIGS. 1 and 2) having a second inner diameter 28 proximate the open neck 26. The second inner diameter 28 at the open neck 26 of the outer container 12 is smaller than the first inner cross-section 24 at the bottom 22 of the outer container 12.

In other embodiments such as that shown in FIGS. 3-4, the bottom shape of the container 12 may be a rectilinear shape to maximize throughput during the lyophilization process. The wall 20 of the outer container 12 having a rectilinear shape also transitions and tapers to form a cylindrical open neck top 26 (see FIGS. 3 and 4) having a second inner diameter 28 proximate the open neck 26. Again, the second inner diameter 28 at the open neck 26 of the outer container 12 is smaller than the first inner cross-section 24 at the bottom 22 of the outer container 12.

The first straining basket 14 has a generally vertical cylindrical wall 30 and a bottom, such as the mesh floor 32 as shown most clearly in FIGS. 1-3, extending across the entire bottom 34 of the first straining basket 14. The wall 30 and mesh floor 32 form a graft holding region 35 in the first straining basket 14, proximate to the mesh floor 32. The wall 30 of the first straining basket 14 has a generally unform outer diameter 36 which slidingly fits within the smaller second inner diameter 28 of the outer container 12. The first straining basket 14 also has a generally uniform inner diameter 37 and locking tabs 38a, 38b extending inward proximate to the open top 39 of the first straining basket 14.

The second straining basket 16, which is the compression feature of this first component 10 of the packaging system, has a generally vertical cylindrical wall 40, a bottom, such as the mesh floor 42 shown in FIGS. 1 and 2, extending across the entire bottom 44 of the second straining basket 16. The mesh floors 32, 42 of the first and second straining baskets 14, 16, respectively, are the fluid separation features of this first component 10 of the packaging system. The wall 40 and mesh floor 42 form a reservoir region 45 at the bottom 44 of the second straining basket 16, proximate to the mesh floor 42. The wall 40 of the second straining basket 16 has a generally unform outer diameter 46 which slidingly and nestingly fits within the inner diameter 39 of the first straining basket 14.

The wall 40 of the second straining basket 16 has a top edge 41 and a planar handle 17 is attached to opposite sides of the wall 40 proximate the top edge 41. The wall 40 of the second straining basket 16 has oppositely positioned vertical indented channels 43a, 43b, which are sized, shaped and positioned to align with the locking tabs 38a, 38b of the first straining basket 14 and allow sliding assembly of the second straining basket 16 inside the first straining basket 14, using the handle 17.

When the second straining basket 16 is rotated within the first straining basket 14 using the handle 17, the top edge 41 of the wall 40 of the second straining basket 16 slides underneath the locking tabs 38a, 38b, so that when the handle 37 is lifted vertically upward the first and second straining baskets 14, 16 are locked and lifted together, with the graft G held or contained in the graft holding region 24 between the mesh floors 32, 42 of the first and second straining baskets 14, 16, respectively.

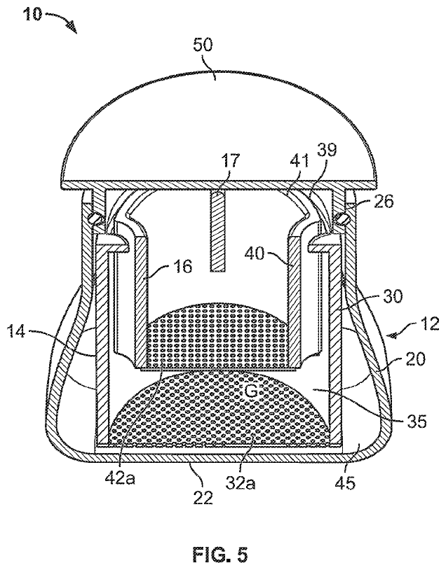

As shown in FIG. 5, as compared to FIGS. 1-3, the size of the mesh (i.e., the size of the mesh openings) of the mesh floors 32, 32a, 42, 42a of the first and second straining baskets 14, 16, respectively, is not particularly limited and may be selected based on the type and composition of graft with which the first component 10 will be used. The mesh size should generally be selected to retain as much of the graft as possible during rehydration, compression, and fluid separation, especially where the graft comprises pieces which are not cohesive when contacted with fluid. Furthermore, while the same mesh size is generally used for each of the mesh floors 32, 42, 32a, 42, this is not required and the mesh sizes may be different for the mesh floors 32, 32a, 42, 42a of the first and second straining baskets 14, 16, respectively.

Additionally, it should be understood that the mesh floors 32, 42, 32a, 42a, do not have to be integrally formed or permanently attached to the first and second straining baskets. Rather, (although not shown per se) the walls 30, 40 of the first and second straining baskets 14, 16 may be constructed with a lip, annular shelf, clip, and or other retaining feature proximate the bottom which would securely hold a separate mesh piece (or other bottom structure with fluid communication means as described below) thereon or therein for providing the required fluid communication. Furthermore, more than one mesh piece may be used or added (e.g., layered) with one another to provide the degree of fluid communication and flow which is desired or sufficiently effective.

For example, first and second straining baskets 14, 16 having mesh floors 32, 42, respectively, with larger sized mesh openings may be more suitable and effective when rehydrating grafts comprising larger pieces (i.e., blocks, layers, chunks, fibers, particles, etc.) or which maintain their cohesiveness during rehydration. On the other hand, when the graft comprises smaller pieces (i.e., chunks, fibers, particles, etc.), or the graft is not sufficiently cohesive during rehydration to avoid separation or disintegration of its pieces (or both), the mesh size of the mesh floors 32, 42 of the first and second straining baskets 14, 16, respectively, should be smaller, which means small enough to retain the pieces of the graft in the event they do separate or disintegrate during rehydration.

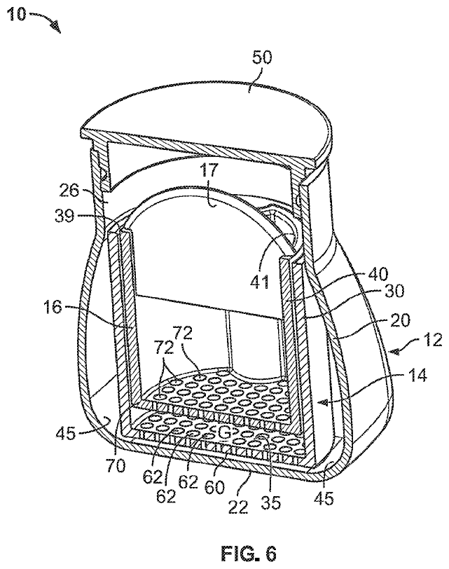

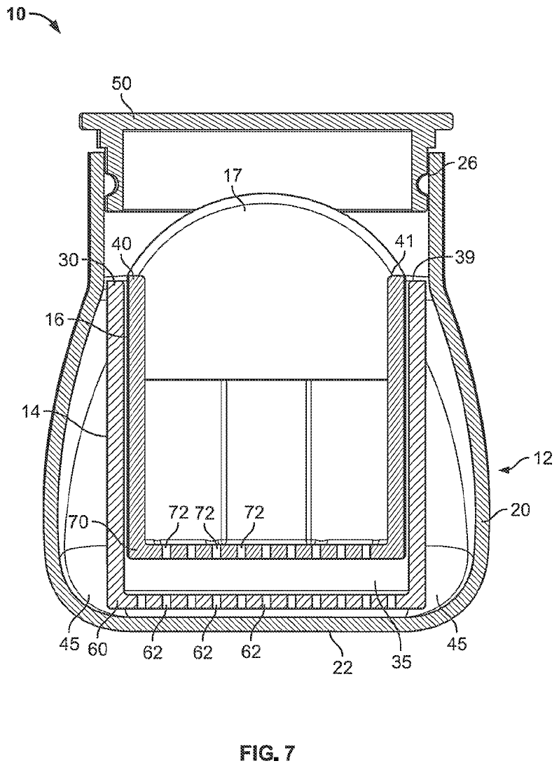

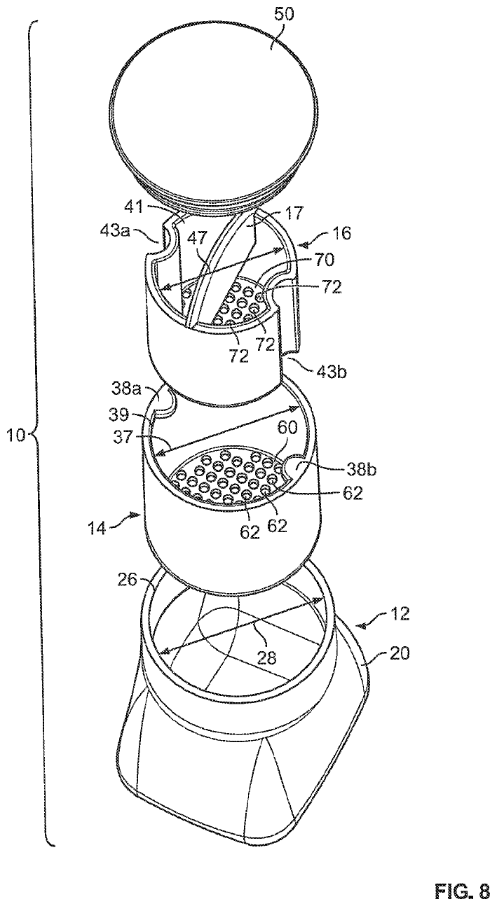

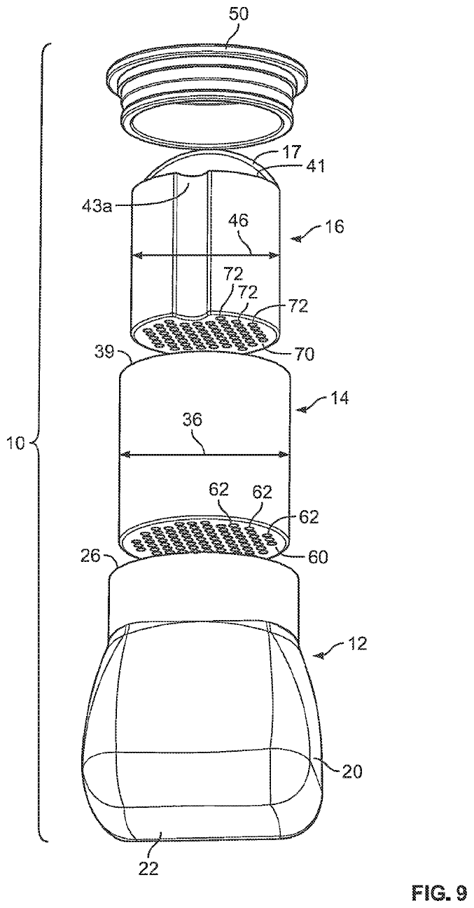

As shown in FIGS. 6-9, in some modified versions of the first component 10, instead of having mesh floors 32, 42 (e.g., shown in FIGS. 1-3 and 5), one or both of the first and second straining baskets 14, 16, respectively, may have a floor with a plurality of apertures or other structure that would allowing escape of fluid therethrough and provide structure for applying compressive force without allowing passage of graft material therethrough or mechanical failure of the bottom floor 32, 42, 60, 70 of the first and second straining baskets 14, 16.

For example, while all of the other elements of the first component 10 remain essentially the same in structure, function, and operation as described above for the embodiment of FIGS. 1-4, FIGS. 6-9 show the first straining basket 14 having a floor 60 with a plurality of apertures 62 therethrough. Similarly, FIGS. 6-9 show the second straining basket 16 having a floor 70 with a plurality of apertures 72 therethrough. The same considerations, options, and factors as discussed above for selecting mesh size for the mesh floors 32, 42 would also apply to selecting the sizes and shapes of the apertures 62, 72 of the floors 60, 70 in the modified versions of the first component 10 and first and second straining baskets 14, 16 shown in FIGS. 6-9. It should be understood that the apertures 62, 72 need not be circular (i.e., having a circular cross-section), but rather, they may each be elongated slots (linear, curved, or a combination thereof), rectilinear (e.g., squares, rectangles, etc.), S-shaped, Z-shaped, star-shaped, or any other shape which is believed to provide the desired fluid flow, while retaining the graft in the graft holding region 35.

Another advantageous modification for the first and second straining baskets 14, 16 might include constructing the walls 30, 40, respectively, or portions thereof, with material or features which provide fluid communication. For example, without limitation, the walls 30, 40 could be made of or include mesh, apertures (having cross-sectional openings which are round, rectilinear, elongate, irregular, etc., and combinations thereof), and other materials or features which provide space separation but also allow passage of fluid therethrough. Additionally, the mesh floors 32, 42 could include one or more apertures as described above for allowing greater fluid flow through the mesh floor 32, 42 than the mesh alone would allow.

As shown more clearly in each of FIGS. 1, 2, and 5-7, the outer container 12 includes an extended portion of the reservoir region 45 of the first component 10, located in the larger inner cross-section 24 portion of the wall 20 of the outer container 12, proximate to the container bottom 22. The solid bottom 22 of the outer container 12 may have a rectilinear shape to maximize throughput during the lyophilization process, or any other production process, as well providing stability of the first component 10, such as during handling, storage, staging (e.g., interim storage or holding periods), and shipping.

The second straining basket 16 has a generally uniform inner diameter 47. As shown in FIG. 1, so that when the first and second straining baskets 14, 16 are assembled in nested fashion, the mesh floor 42 of the second straining basket 16 forms the upper boundary of the graft holding region 35, while the mesh floor 32 of the first straining basket 14 forms the lower boundary of the graft holding region 35.

The assembly and operation of the first component 10 for rehydrating a graft will now be provided. While the following description is provided with reference to the embodiment of the first component shown in FIGS. 1-4, it should be understood that the modified embodiments of the first component 10 shown in FIGS. 5 and 6-9 are operable in a similar manner.

For operation of the first component 10 of the multi-component packaging system, a graft G (not shown per se, but location indicated in FIG. 1) is loaded in between the mesh floors 32, 42 of the first and second straining baskets 14, 16, respectively. Rehydration fluid (not shown) is added to the open neck 26 of the rigid outer container 12. It is noted that the open neck 26 of the outer container 12 is the previously described second opening of the first component 10 which allows fluid communication between the reservoir region 45 and the external environment. A lid 50 (e.g., a press-fit plastic closure, a screw top plastic cap, or a stopper) is provided to sealingly close the open neck 26 of the outer container 12. Alternatively, a peelable heat-sealed lid can be applied as a lid closure for the outer container 12. The lid may be configured with venting features that enable venting during lyophilization and following that process the lid may be fully closed and sealed.

In its fully assembled state shown in FIG. 1 (but with a graft in the graft holding region 35 between the mesh floors 32, 42), the first component 10 may be inverted to strain while simultaneously gently squeezing the first and second straining baskets 14, 16 together to apply compressive force to the graft G and separate and remove as much fluid as possible. This inversion and compression will displace fluid from the graft holding region 35 to the reservoir region 45. Additionally, if the lid 50 is removed from the outer container 12, the first and second straining baskets 14, 16 may also be lifted from the container while simultaneously squeezing to apply compressive force to the graft G between the mesh floors 32, 42 to separate and remove additional fluid from the graft G, whereupon that additional fluid is displaced from the graft holding region 35 into the extended reservoir region 45 at the bottom 22 of the outer container 12. A user may also lift the first and second straining baskets 14, 16 with the graft G held between the mesh floors 32, 42, followed by squeezing and using the container 20 as a reservoir to receive displaced fluid or not.

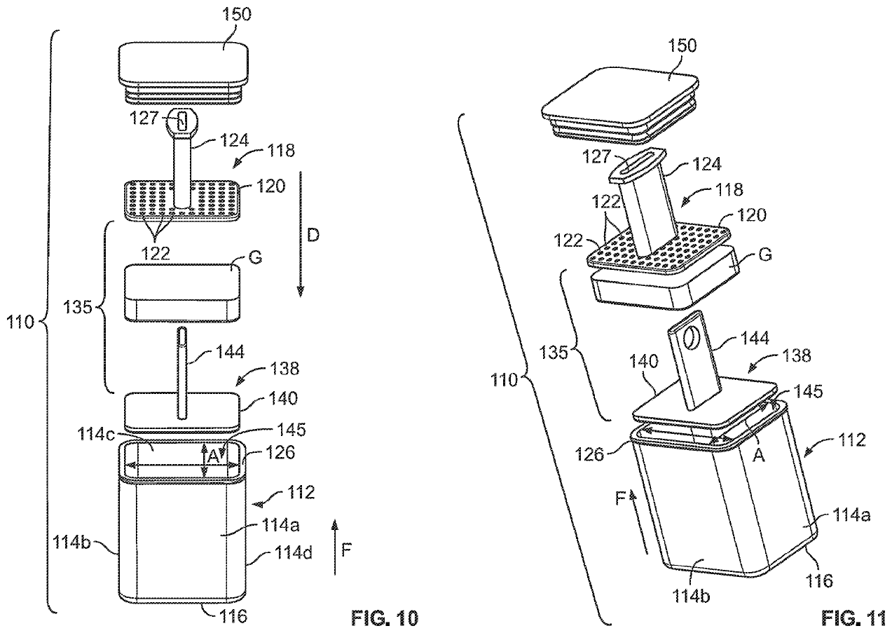

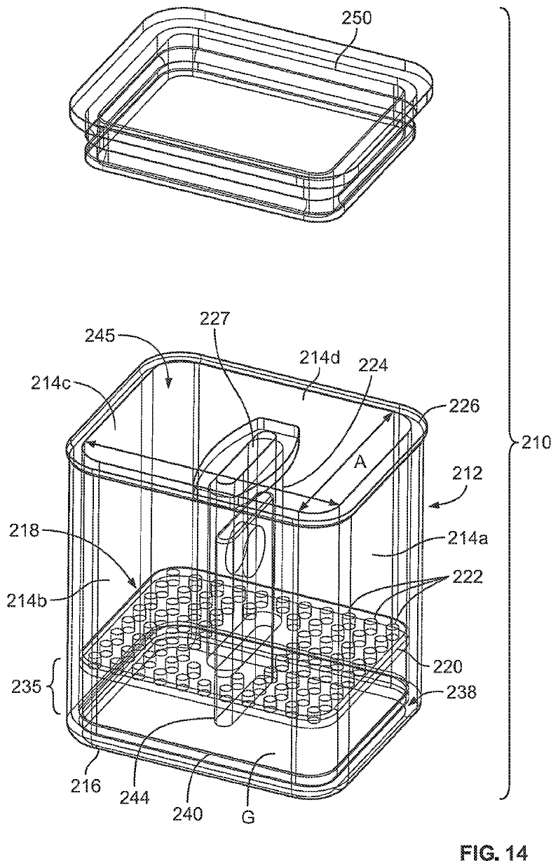

In other exemplary embodiments shown in FIGS. 10-19, a first component 110, 110′, 210 of a multi-component packaging system for grafts includes one or more plunger elements as the compression features. More particularly, as will be described in detail hereinafter, FIGS. 10-12 provide several views of one exemplary embodiment of the first component 110 which includes first and second plungers, while FIG. 13 shows and alternative embodiment of the first component 110′ which includes only a single plunger. Finally, FIGS. 14-19 provide several views of a modified first component 210 which includes first and second plungers like the first component 110 of FIGS. 10-12, but is made of a translucent or transparent material to enable viewing of a graft while being rehydrated during operation of the first component 210.

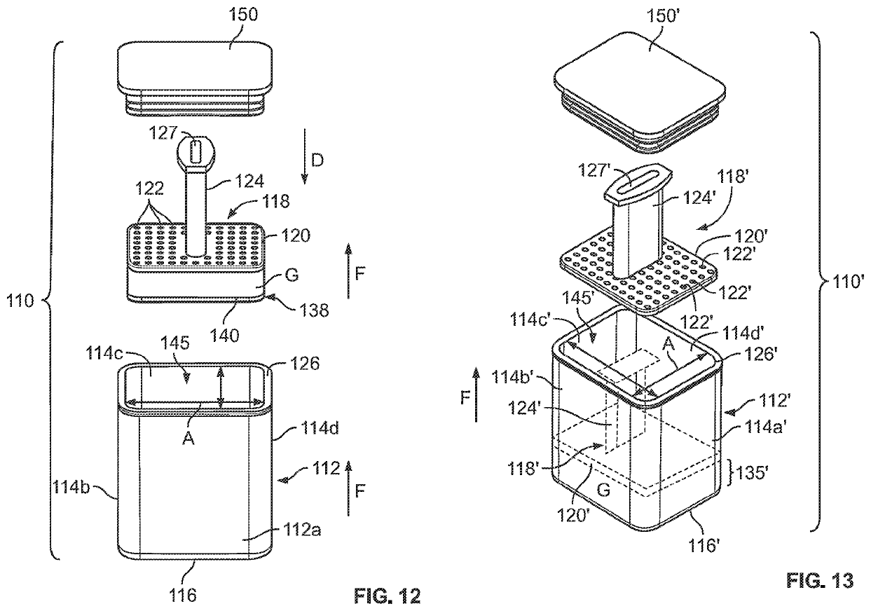

Turning now to FIGS. 10-12, several views are provided of a first exemplary embodiment of a packaging system, in which the first component thereof 110 includes first and second plungers 118, 138 as compression features, in accordance with the invention described and contemplated herein. More particularly, FIGS. 10 and 11 each provide an exploded perspective view of the first component 110 having first and second plungers 118, 138. Each of FIGS. 10-12 shows the position of a graft G to be rehydrated relative to the elements of the first component 110. FIG. 12 specifically provides a partially assembled perspective view (see FIG. 12) in which the first and second plungers 118, 138 are assembled together with a graft positioned in between them.

In addition to the first and second plungers 118, 138, the first component 110 of this plunger embodiment comprises a rigid container 112 having a reservoir region 145 which defined by side walls 114a, 114b, 114c, 114d and a solid bottom 116. The reservoir region 145 has an inner cross-sectional area A bounded by the side walls 114a, 114b, 114c, 114d. As described in detail later, the first and second plungers 118, 138 and a graft G positioned therebetween are all received and held in the reservoir region 145 to facilitate both rehydrating the graft G by contacting it with a rehydrating fluid (not shown per se), as well as separation and removal of excess rehydration fluid from the graft G.

An open top 126 provided on the rigid container 112 (which is analogous to the open neck 26 of the outer container 12 of the above-described embodiment of a first component 10 of FIGS. 1-9) allows rehydrating fluid to be provided to the reservoir region 145 from the external environment. The opening 126 of the container 112 has the inner cross-sectional area A which is defined by the side walls 114a, 114b, 114c, 114d. A lid 150 (e.g., a press-fit plastic closure, a screw top plastic cap, or a stopper) is provided to cover and sealingly close the open top 126 of the rigid container 112. Alternatively, a peelable heat-sealed lid (not shown) can be applied as a lid closure 250 for the container 212.

As shown in each of FIGS. 10-12, the first plunger 118 has a compression plate 120 with a plurality of apertures 122 therethrough. The first plunger 118 also has an elongate vertically-oriented mating element or handle 124 with a vertically oriented opening 127 therethrough to allow assembly with the second plunger 138, as will be described below. The compression plate 120 of the first plunger 118 is sized and shaped to fit slidingly in the container 112 and has a size and shape which extends across the aforesaid inner cross-sectional area A of the container 112. In this case, the first plunger 118 and its compression plate 120 with apertures 122 serve as both the compression feature and the fluid separation feature of this embodiment of the first component 110 of the packaging system.

The second plunger 138 includes a solid base plate 140 and a handle 144 extending vertically from an approximate center of the top surface of the solid base plate 140 (see FIGS. 10-11) for manipulating the plunger 138 in the reservoir region 145 of the container 112. The handle 144 of the second plunger 138 is sized and shaped to be received slidingly through the opening 127 of the mating element 124 of the first plunger 118. This arrangement facilitates alignment and cooperation of the first and second plungers 118, 138 with one another. In the foregoing alignment of the first and second plungers 118, 138, a graft holding region 135 is provided between the compression plate 120 of the first plunger 118 and the solid base plate 140 of the second plunger 138.

As is clear from FIGS. 10-12, the compression plate 120 of the first plunger 118 has dimensions coextensive with the solid base plate 140 of the second plunger 238, the bottom 116 of the container 112, and the inner cross-sectional area A of the container 112. As in some other embodiments, the dimensions of the rigid container 112 are selected based on the size of the graft(s) expected to be rehydrated using the first component 110 and the expected or desired maximum amount of rehydration fluid that is to be separated and removed from the rehydrated graft G.

FIG. 12 shows the first and second plungers 118, 138 oriented and assembled together with a graft G positioned in the graft holding region and contacting both the compression plate 120 and the solid base plate 140 of the first and second plungers 118, 138, respectively. Notwithstanding the arrangement of elements in FIG. 12, when the first component 110 is assembled for actual use, the second plunger 138 typically is inserted into the container 112 first, with its solid base plate 140 lying flat across the bottom 116 of the container 110, followed by placement of the graft G on over the solid base plate 140 and addition of rehydration fluid (not shown per se) to the reservoir region 145, via the open top 126 of the container 112. Then, the first plunger 118 is positioned in the reservoir region 145, over the graft G and aligned so that the handle 144 of the second plunger 138 is received within the vertically-oriented opening 127 of the handle 124 of the first plunger 118.

After sufficient time has passed for the graft G to rehydrate, excess fluid is separated and removed from the graft G while still held in the reservoir region 145, between the plates 120, 140 of the first and second plungers 118, 138, respectively, as follows. The first plunger 118 is pressed down (see arrow D in FIGS. 10 and 12) to cause the compression plate 120 to apply pressure to the graft G and thereby squeeze and separate excess fluid from the graft G. The compressive force applied by the compression plate 120 also displaces the separated fluid through the apertures 122 and up (see arrow F in FIGS. 10-12 showing the direction of fluid movement upon application of compression by the compression plate 120) out of the graft holding region 135 (between the compression plate 120 and the solid base plate 140) and into the reservoir region 145 (i.e., above the compression plate 120), from where it can be poured or decanted off, or absorbed with gauze and removed through the open top 126 of the container 112.

The assembled first and second plungers 118, 138 of this embodiment may be lifted up and out of the reservoir region 145 of the container 112, together with the rehydrated graft G between the compression plate 120 and base plate 140, and out of the reservoir region 145 via the open top 126. This arrangement facilitates recovery of the entire rehydrated graft G from the container 112, after rehydration and fluid separation from the graft G, without the need to scrape or otherwise subsequently collect portions of the graft G which might otherwise remain in the reservoir region 145 in the absence of the second plunger 138 and its solid base plate 140.

FIG. 13. shows a modified first component 110′ of the packaging system, as described and contemplated herein, that is similar to the first component 110 of FIGS. 10-12, but includes only a single plunger 118′. All of the other elements of the first component 110′ of FIG. 13 are the same in structure, function, and operation as the first component 110 described above in connection with FIGS. 10-12 and, therefore, have been given the same reference numbers with the addition of a prime mark or single quotation mark (′). In the absence of a second plunger (138 of the previously described embodiment of FIGS. 10-12), the assembly and operation of this modified first component 110′ is a bit different, as follows.

The plunger 118′ still includes a compression plate 120′ with a plurality of apertures 122′ therethrough. The compression plate 120′ is sized and shaped to fit slidingly in the container 112′ and having a size and shape which extends across the inner cross-sectional area A of the container 112′. The plunger 118′ has a handle 124′ extending vertically from the top surface of the compression plate 120′ for manipulating the plunger 118′ in the container 112′. An optional vertically oriented opening 127′ may be provided through the handle 124′.

In the embodiment of FIG. 13, when the plunger 118′ is in a position (indicated by dotted lines in FIG. 13) intermediate (in between) its fully withdrawn position (plunger 118′ in solid lines in FIG. 13) and its fully inserted position (not shown but in which the compression plate 120′ would lie over and in contact with the bottom 116′ of the container 112′), a space or open volume is formed in the container, 112′ between the compression plate 120′ and the bottom 116′ of the container 112′. This open volume is the graft holding region 135′ for this embodiment of the packaging. The plunger 118′ and its compression plate 120′ with apertures 122′ serve as both the compression feature and the fluid separation feature for this embodiment.

In operation, the plunger 118′ is lifted until the compression plate 120′ is entirely out of the container 112′ (plunger 118′ in solid lines in FIG. 13). A graft (indicated by G in FIG. 13) is placed on the solid bottom 116′ of the container 112′, and then the compression plate 120′ of the plunger 118′ inserted back into the container, through the open top 126′, and pressed down to apply compressive force to the graft G. When the compression plate 120′ of the plunger 118′ is pressed onto the graft G, fluid (not shown per se) is separated from the graft G and displaced up through the plurality of apertures 122′ (see arrow F in FIG. 13) and into the space remaining above the compression plate 120′ but still within the container 112′, that space being the reservoir region 145′ for receiving fluid separated and removed from the graft G and displaced from the graft holding region 135′ by compression forces applied through the compression plate 120′ of the plunger 118′. Any fluid which is displaced from the graft holding region 135′ underneath the compression plate 120′ and up into the reservoir region 145′ above the compression plate 120′ may be removed from the container 112′ via the open top 126′, by pouring, decanting, or using gauze to absorb and remove such fluid. If any significant portion of graft material remains in the container 112′ after removal of the plunger 118′ and graft G from the graft holding region 135′ it may be collected by scraping or scooping the remaining graft material from the bottom 116′ of the container 112′.

As mentioned above, FIGS. 14-19 show several views of another modified first component 210, in accordance with the packaging system described and contemplated herein. This modified first component 210 is similar to that shown in FIGS. 10-12 and discussed above, but is made of a translucent or transparent material to enable viewing of a graft G being rehydrated during operation of the first component 210.

All of elements of the first component 210 of FIGS. 14-19 are the same in structure, function, and operation as the first component 110 described above in connection with FIGS. 10-12. Accordingly, the elements of this modified first component 210 have reference numbers corresponding to those used in FIGS. 10-12 but increased by 100 (e.g., whereas FIGS. 10-12 shown the first component 110, FIGS. 14-19 show the modified first component 210). The assembly and operation of this modified first component 210 is the same as for the first component 110 of FIGS. 10-12 and is repeated below for clarity and completeness.

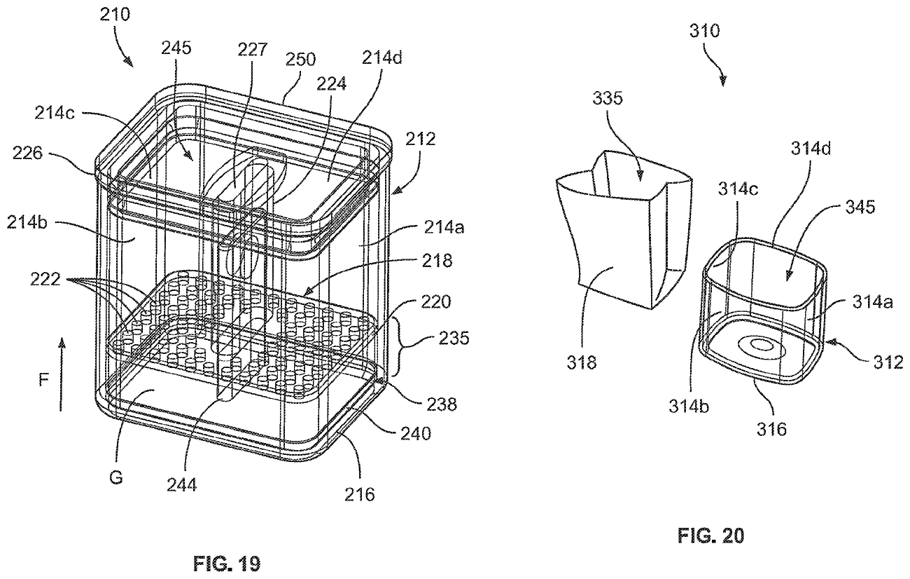

FIG. 14 provides a perspective view of the modified first component 210 made of translucent material and in a nearly completely assembled state with a graft G positioned therein, but not yet covered with the lid 250. FIG. 19 provides a perspective view of the modified first component 210 of FIG. 14 in its completely assembled state, with a graft G positioned therein and the 250 thereon.

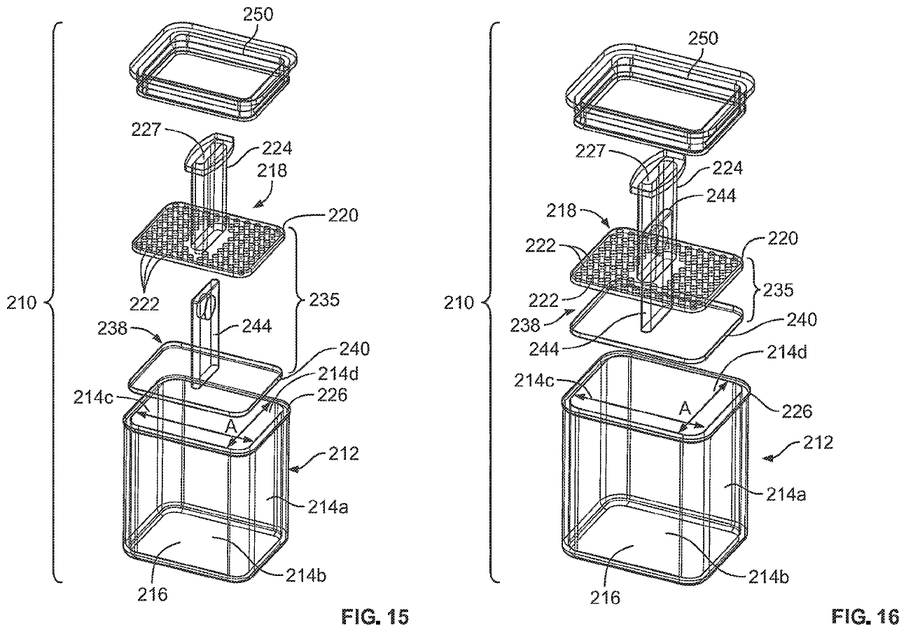

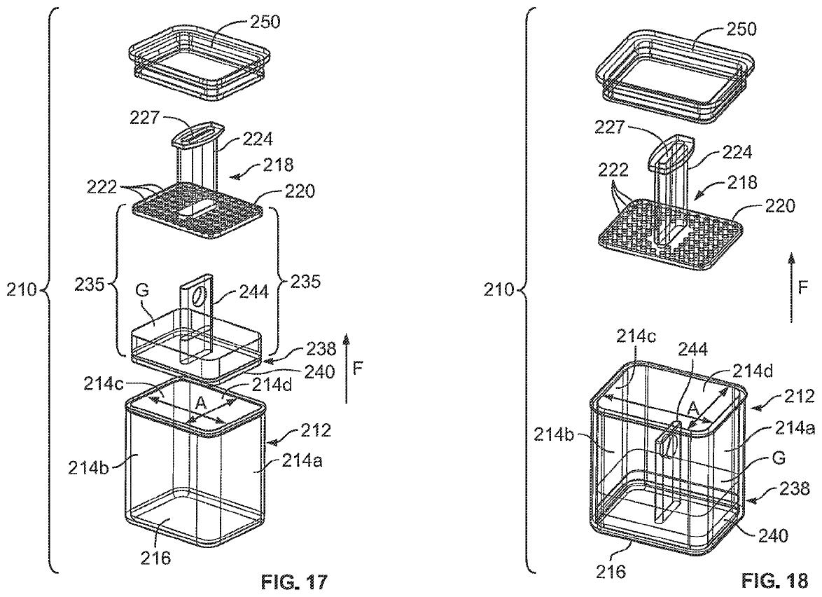

FIGS. 15-18 provide several exploded perspective views of the modified first component 210 in various stages of assembly. More particularly, FIG. 15 is an exploded top perspective view of the modified first component 210, while FIG. 16 is an exploded top perspective view of the modified first component 210, showing how the first and second plungers 218, 238 fit slidingly together. Furthermore, FIG. 17 is an exploded top perspective view of the modified first component 210, showing the arrangement of elements and a graft G mounted on the second plunger 238, and FIG. 18 shows the modified first component 210 of FIG. 17, but with the second plunger 238 and graft G mounted thereon both positioned in the container 212.

Generally, the modified translucent first component 210 comprises a rigid container 212 having an inner cross-sectional area A bounded by side walls 214a, 214b, 214c, 214d and a solid bottom 216. This modified first component 210 comprises first and second plungers 218, 238, which cooperate to separate fluid from a graft and at least partly facilitate recovery and removal of the rehydrated graft material from the container 212 after fluid separation and removal has been accomplished.

An open top 226 of the container 212 allows rehydrating fluid to be delivered to the reservoir region 245 from the external environment. A lid 250 (e.g., a press-fit plastic closure, a screw top plastic cap, or a stopper) is provided to sealingly close the open top 226 of the container 212. Alternatively, a peelable heat-sealed lid (not shown) can be applied as a lid closure 250 for the container 212.

As shown most clearly in each of FIGS. 15-18, the first plunger 218 has a compression plate 220 with a plurality of apertures 222 therethrough. The first plunger 218 also has an elongate vertically-oriented mating element 224 with an opening 227 therethrough for a purpose described below. The compression plate 220 of the first plunger 218 is sized and shaped to fit slidingly in the container 212 and has a size and shape which extends across the inner cross-sectional area A of the container 212. In this case, the first plunger 218 and its compression plate 220 with apertures 222 serve as both the compression feature and the fluid separation feature of this embodiment.

A second plunger 238 is also provided and includes a solid base plate 240 and a handle 244 extending vertically from an approximate center of the top surface of the solid base plate 240 for manipulating the plunger in the container 212. In addition, as shown partially in FIG. 16, the handle 244 of the second plunger 238 is sized and shaped to be received slidingly through the opening 226 of the mating element 224 of the first plunger 218. This arrangement facilitates alignment and cooperation of the first and second plungers 218, 238 with one another.

As is clear from FIGS. 14-19, the solid base plate 240 of the second plunger 238 has dimensions coextensive with the compression plate 220 of the first plunger 218, the bottom 216 of the container 212, and the inner cross-sectional area A of the container 212. As in some other embodiments, the dimensions of the rigid container 212 are selected based on the size of the graft expected to be rehydrated using the modified first component 210 and the expected or desired maximum amount of fluid that can be separated and removed from a graft G. When the first component 210 is assembled, the second plunger 238 is inserted into the container 212 first, with its solid base plate 240 lying flat across the bottom 216 of the container 210.

As can be understood from FIGS. 14-19, when this modified first component 210 is assembled and operated, a graft holding region 235 is formed between the compression plate 220 of the first plunger 218 and the solid base plate 240 of the second plunger 238. The first plunger 218 is pressed down to cause the compression plate 220 to apply pressure to the graft G to squeeze and separate fluid from the graft G. The compressive force applied using the compression plate 220 also displaces the separated fluid through the apertures 222 and up (see arrow F in each of FIGS. 17-19) into the reservoir region 245 (i.e., above the compression plate 220, in the container 212), from where it can be poured or decanted off, or absorbed with gauze and removed through the open top 226 of the container 212.

Like the first component 110 of the packaging system described above and shown in FIGS. 10-12, the assembled first and second plungers 218, 238 of this embodiment of the first component 210 may be lifted up and out of the container 212 together, through the open top 226, with the graft G between the compression plate 220 and base plate 240, thereby providing easier recovery of substantially the entire rehydrated graft G from the container 212 after fluid separation from the graft G.

In still another exemplary embodiment of packaging for grafts in accordance with the present disclosure, FIGS. 20-22 provide several views of a first component 310 which generally comprises a rigid container 312 and a mesh pouch 318. FIG. 20 shows the rigid container 312 and mesh pouch 318 prior to assembly and both being empty. The mesh pouch 318 is sized and shaped to be received within the container 312 and has an interior space which is the graft holding region 335 (see FIG. 20). Additionally, the mesh pouch 318 comprises a mesh size which allows fluid to pass therethrough, into and out of the graft holding region 335 therein.

With reference to FIG. 20, the rigid container 312 has sidewalls 314a, 314b, 314c, 314d and a solid bottom 316 which form an interior space which is the reservoir region 345 for receiving rehydrating fluid separated from the graft G (see, e.g., FIG. 10) and displaced from the graft holding region 335, through the mesh pouch 318. Although the container 312 is shown in FIG. 20 as having a basically rectilinear shape, the shape of the container 312 is not particularly limited. Rather, the container 312 should simply be sized to receive rehydration fluid and substantially the entire mesh pouch 318 in the reservoir region 345. In use, the graft G is typically placed inside the graft holding region 335 of the mesh pouch 318.

In use, rehydration fluid RF (not shown per se) may be added to the container 312, either before or after the mesh pouch 318 is placed in the container 312. The mesh pouch 318, with the graft therein, is then placed into the reservoir region 345 of the container 312 and submerged in the rehydration fluid, which passes through the mesh pouch 318 and into the graft holding region 335 to contact and hydrate the graft G therein. Compressive force may be applied to the mesh pouch 318 and the graft G therein, by manually (e.g., using two or more fingers), pressing or pinching on opposite sides of the mesh pouch 318 and sliding down the mesh pouch 318 to squeeze the mesh pouch 318 and the graft G disposed therein.

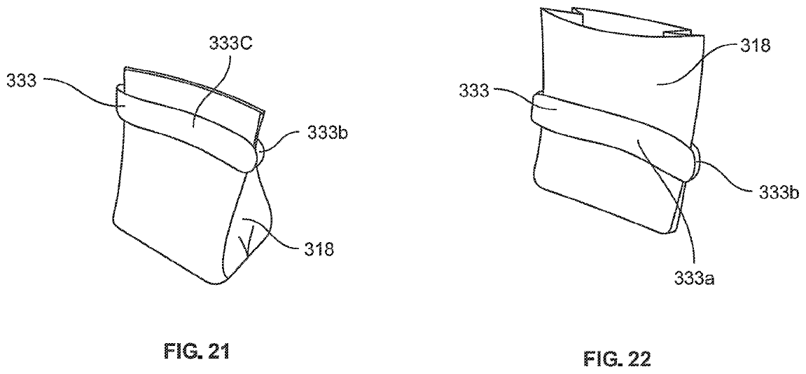

As shown in FIGS. 21 and 22, a compression element, such as a compression clip 333, may be provided to assist with applying compression to the mesh pouch 318 and graft G therein. As shown in FIGS. 21 and 22, such a compression clip 333 could be sized and shaped to tightly and slidingly straddle both sides of the mesh pouch 318. For example, the compression clip 333 may include two bars 333a, 333b (bar 333a is more clearly visible) which are disposed on opposite sides of the mesh pouch 318 for applying compressive force as the compression clip 333 is slidingly moved downward, from an upper portion of the mesh pouch 318 (FIG. 21) to a lower portion of the mesh pouch 318 (FIG. 22). This operation of the compression clip 333 enables squeezing the mesh pouch 318 to force fluid to separate from the graft G and into the graft holding region 335 inside the mesh pouch 318, and then be displaced from the graft holding region 335, through the mesh pouch 318, and into the reservoir region 345 of the container 312. After such separation and removal of excess fluid, the compression clip 333 may be removed from the mesh bag 318 and the rehydrated graft removed from the mesh pouch 318.

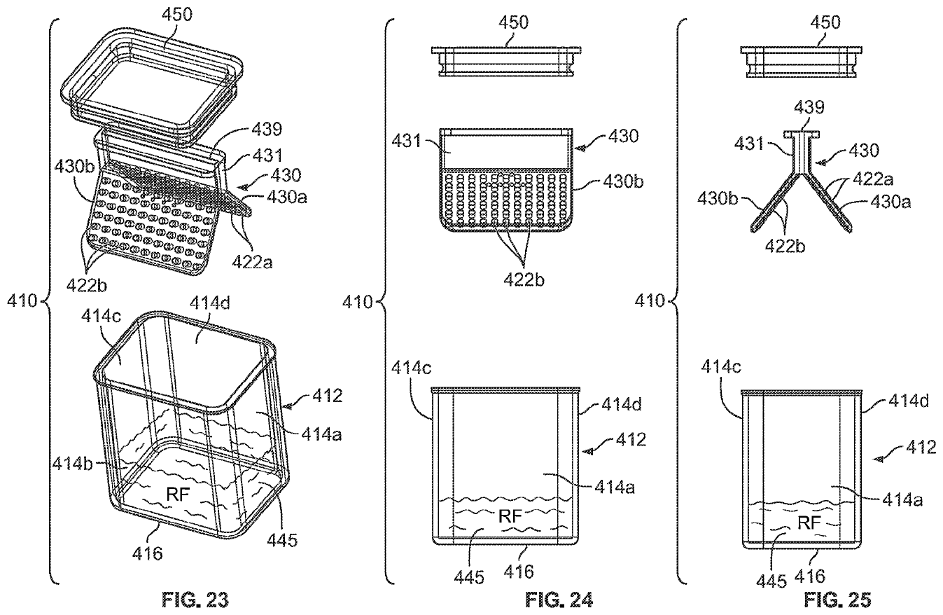

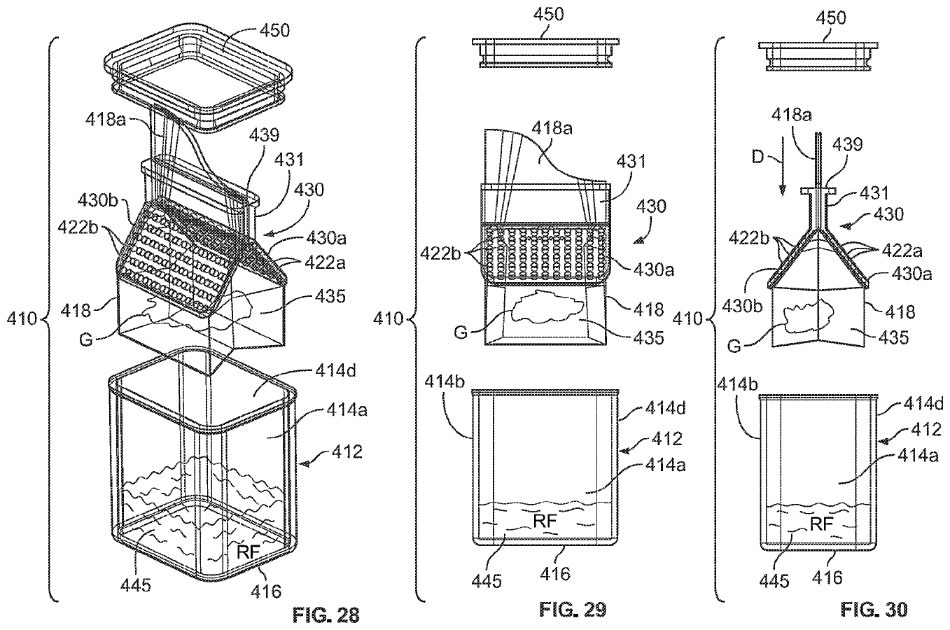

FIGS. 23-30 provide several views of a modified first component 410 which is somewhat similar to the first component 310 described above and shown in FIGS. 20-22, but which includes a modified compression clip 430 and a lid 450 for covering the container 412. Accordingly, the rigid container 412 of this embodiment also has sidewalls 414a, 414b, 414c, 414d and a solid bottom 416 which form an interior space which is the reservoir region 445 for receiving rehydrating fluid separated from the graft G and displaced from the graft holding region 435, through the mesh pouch 318. Although the container 412 is shown in FIGS. 23-30 as having a basically rectilinear shape, the shape of the container 412 is not particularly limited. Rather, the container 412 should simply be sized to receive rehydration fluid and substantially the entire mesh pouch 418 in the reservoir region 445.

In use, the graft G is placed inside the graft holding region 435 of the mesh pouch 418 either before or after the mesh pouch 418 is placed in the reservoir region 445 of the container 412, but always prior to assembly of the mesh bag 418 with the compression clip 430.

Like the first component of FIGS. 20-22, this modified first component 410 may include a compression element, such as the compression clip 430 shown in FIGS. 23-30, to assist with applying compression to the mesh pouch 418 and graft G therein. As shown in the several views provided in FIGS. 23-30, the compression clip 430 tightly and slidingly straddle both sides of the mesh pouch 418 to assist with squeezing its contents to separate fluid from the graft G and the graft holding region 435 inside the mesh pouch 418, and displace separated fluid from the graft holding region 435, through the mesh pouch 418 and into the reservoir region 445 of the container 412.

In some embodiments such as that provided in FIGS. 23-30, the compression clip 430 may have wings 430a, 430b disposed on, and in contact with, opposite sides of the mesh pouch 418. Each of the wings 430a, 430b has a plurality of perforations 422a, 422b, respectively, to allow fluid displaced from the graft holding region 435, inside the mesh pouch 418, to the reservoir region 445 of the container 412 above the wings 430a, 430b. The wings 430a, 430b provide greater surface area to contact opposite sides of the mesh pouch 418, which consequently provides more efficient compression to separate and displace fluid as described above, from the graft and graft holding region 435, through the mesh pouch 418 and received in the reservoir region 445 of the container 412.

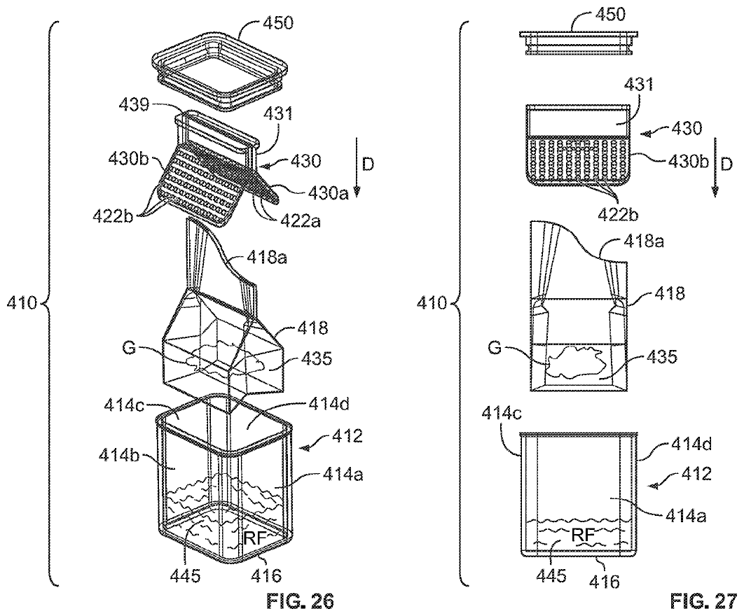

As the compression clip 430 is slidingly moved from the top to the bottom of the mesh pouch 418 (direction of movement of compression clip 430 shown by arrow D in FIGS. 26, 27, and 30), with a graft G therein, the volume of the graft holding region 435 is reduced and compressive force is applied to opposite sides of the mesh pouch 418 and the graft G therein. The compression clip 430 may include a bridge 431 with an opening 439 therethrough for receiving an upper portion 418a of the mesh pouch 418 therethrough which enables grasping the upper portion 418a of the mesh pouch 418 to assist in moving the compression clip 430 downward along the mesh pouch 418.

General operation of the first component 410 of this embodiment of the packaging is as follows, although it should be understood that variations in the following procedure are possible and well within the contemplated and effective various methods of operating the modified first component 410. After a graft G is placed in the mesh pouch 418 (i.e., in the graft holding region 435), and the mesh pouch 418 has been submerged and soaked in the rehydration fluid RF, for example in the reservoir region 445 of the container 412, for a period sufficient for the graft G to absorb at least a portion of the rehydration fluid RF, the compression clip 430 is slid downward, i.e., in the direction of the arrow D in FIGS. 26, 27, and 30, thereby applying compressive force to both sides of the mesh pouch 418 to separate fluid from the graft G and into the graft holding region 435, and then displace it from the mesh pouch 418 to the reservoir 445 of the container 412. After removal of the compression clip 430, the mesh pouch 418 should be peelable/easily opened so a user can access the rehydrated graft G easily after performing the foregoing fluid separation and displacement method.

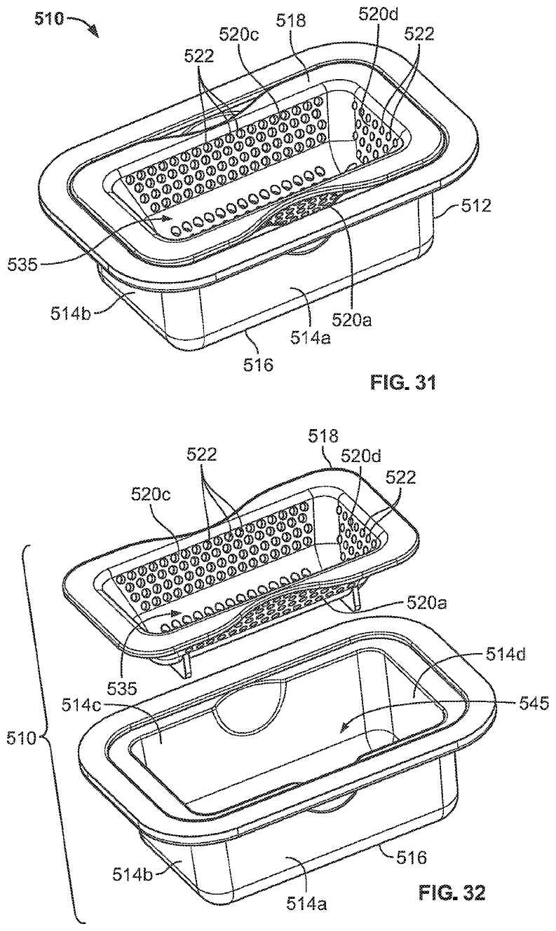

FIGS. 31-32 show still another exemplary embodiment of a multi-component packaging system according to the present disclosure that comprises a first component 510 which includes a rigid tray 512 having sidewalls 514a, 514b, 514c, 514d and a solid bottom 516 which cooperate to form a reservoir region 545. In this exemplary embodiment, the first component 510 further includes a V-shaped strainer tray 518 with sloping sidewalls 520a, 520b, 520c, 520d which cooperate to form a V-shaped graft holding region 535, and wherein one or more of the sloping sidewalls 520a, 520b, 520c, 520d has a plurality of apertures 522.

In use, a graft (not shown) will be loaded into the V-shaped graft holding region 535. Rehydration fluid is then added directly to the graft in the graft holding region 535, whereupon the graft will absorb at least a portion of the rehydration fluid, while any unabsorbed or excess rehydration fluid is strained or filtered through the apertures of the V-shaped strainer tray 518 and into the reservoir region 535 of the rigid tray 512 positioned underneath the V-shaped strainer tray 518. Compressive force may be applied to the graft while in the V-shaped strainer tray by pressing down on the graft either manually (e.g., using one or more fingers) or with a sterile instrument (e.g., a spatula, spoon, etc.), which compresses the graft, thereby separating fluid from the graft and driving the fluid downward through the apertures of the V-shaped strainer tray 518.

It will be understood that the embodiments of the present invention described hereinabove are merely exemplary and that a person skilled in the art may make variations and modifications without departing from the spirit and scope of the invention. All such variations and modifications are intended to be included within the scope of the present invention.

For example, for any of the above-described embodiments, manual pressure may be applied to any one or more of the packaging components. Without limitation, it is contemplated that applying manual compression or other pressure to one or both of the straining baskets, or to the mesh pouch, in addition to using the devices as described above to apply such compression or pressure, is possible and could be advantageous, for example where operation of the elements of any of the embodiments of the first component alone does not remove enough fluid. In such circumstances, a user may also use their fingers to press on the graft while in the packaging to assist with additional fluid separation and displacement.

### EXAMPLES

### Example 1

Test preliminary concepts for ability to facilitate rehydration, decanting and squeezing bone fiber and qualitatively assessed handling properties of tissues derived matrices in preparation for implanting (medical use).

### Materials and Equipment:

Lyophilized bone tissue derived matrices
Packaging prototypes varying in shape, dimensions, and materials
Mesh of varying materials, dimensions, porosity or opening size
Phosphate Buffered Saline
Scalpel
Forceps
Weigh pans
Basin

### Procedure:

1. Baseline—demonstrated current standard practices observed in clinical use of tissue derived matrices. Transferred dry tissue derived matrix to a container. Added fluid to the container and attempted to decant or strain fluid. Removed tissue derived matrix from the container.
2. Container with perforated lidstock prototype adhered to form a closure—Transferred a dry matrix tissue sample to a container. Added saline to rehydrate and then decanted fluid through the openings of the perforated lidstock. Tissue was removed from the container.
3. Mesh basket prototype with or without compression feature. Filled with dry tissue derived matrix in the basket, placed basket in a container. Added saline to rehydrate. Lifted/removed the basket containing the tissue. For basket with compression feature, applied compression to remove additional fluid from the matrix.
4. Flexible mesh pouch prototype
   a. Placed dry tissue derived matrix into mesh pouch. Placed into container. Added saline to rehydrate. Removed the mesh pouch containing tissue. Repeat with manual wringing or compression to remove additional fluid from the matrix while contained in the mesh.
5. All tissue derived matrices samples were qualitatively assessed and compared for

### Methods:

All tissue derived matrices samples were qualitatively assessed and compared for handling properties as described below.

For all assessments and test methods applied and described herein, the total preparation time using the packaging is defined as: the total amount of time starting from when the user pours fluid used to rehydrate the tissue and until when the tissue is ready to use by the user as noted by appropriate cohesive and moldable properties.
1) amount of fluid visibly separated from the graft using the packaging:
   a) “Good”=more than half of the fluid was removed from the graft
   b) “Poor”=less than half of the fluid was removed from the graft
2) graft handling properties upon removing from the package such as:
   a) “Cohesive”=The tissue was assessed for cohesiveness which is the ability of the tissue to maintain shape and does not fall apart when picked up by the user using instruments, hands or other means.
   b) “Moldable”=The tissue is flexible and can be shaped by the user to meet the shape of the tissue application. Graft can be manually molded into a shape such as ball or cube with smooth edges
3) Condition of the graft upon removing from the package
   a) “Saturated”=upon further compression of the graft, drops or no free flowing fluid
   b) “Oversaturated”=steady stream of free flowing fluid with or without further compression of the graft
4) It was also noted if additional steps were necessary for the rehydrated graft to be considered ready for clinical implant. For example, standard practice has been observed to require end users to decant from the container. Users have been observed to use a surgical towel, sponge, or gauze to cover the container while decanting. The towel, sponge, or gauze acts as a strainer to separate the fluid from the graft. Users have also been observed to decant and then remove graft from the packaging and hold in their hand or wrap a surgical towel, sponge, or gauze around the graft and manually wring/squeeze out additional fluid to make the graft ready to use (as defined by moldable and cohesive above). Users have also been observed to decant and place the surgical towel, sponge, or gauze directly on top of the graft to absorb additional fluid.

### Results:

| | Standard | | | | |
|---|---|---|---|---|---|
| | Practice/ | Perforated | Plunger | Mesh Basket | |
| | baseline | lidstock | with mesh | w/plunger | Mesh Pouch |
| Total estimated | 2 min 45 | 3 min 30 sec | 2 min 30 | 2 min 30 | 3 minutes |
| preparation | seconds | | seconds | seconds | |
| time using the | | | | | |
| packaging | | | | | |
| Visible Fluid | Good (used | Good | Good | Good | Good |
| Separation from | towel in | | | | |
| Graft | addition to | | | | |
| | the packaging | | | | |
| | to strain) | | | | |
| Graft handling | Oversaturated, | Oversaturated, | Saturated; | Saturated; | Saturated; |
| upon removal | not cohesive | not | cohesive; | cohesive; | cohesive; |
| from packaging | or moldable | cohesive or | moldable | moldable | moldable |
| | | moldable | | | |
| Additional | Towel to | manual | None | None | None |
| steps to achieve | absorb | wringing | | | |
| desired graft | additional | | | | |
| handling | fluid | | | | |

### Example 2

(Demonstrated less steps to prepare graft for implantation; Eliminated extra tools in the operating room; Demonstrated good/acceptable handling right out of the package)

Evaluated a prototype container with a basket designed to contain various volumes of the tissue graft and a plunger to apply manual compressive force to facilitate removal of fluid from tissue graft. The basket and plunger were each a single, formed piece with formed openings of various dimensions and shapes to allow fluid to transfer from the container to the tissue graft inside the basket and from the tissue graft inside the basket to the container.

### Materials:

Hydrated bone graft in fiber and particle form

### Procedure:

1. Simulated a potential tissue graft production process:
   a. Placed the basket inside the container
   b. Filled basket with unprocessed bone tissue graft in a fiber and particle configuration
   c. Added preservative solution to the tissue graft.
   d. Placed plunger inside the basket; on top of the tissue graft
   e. Performed the freezing and preservation process of reducing moisture for ambient shelf-life stability
2. Simulated the clinical preparation procedure to ready the graft for implant
   a. Opened the container
   b. Add saline solution to the top of the container to cover the tissue graft
   c. Allow tissue to sit in solution for at least 2 minutes to allow the tissue graft to rehydrate
d. Decant or remove excess solution by using the packaging components (i.e. basket and plunger)
   e. Remove graft from packaging. Perform additional steps if needed to reach a moldable and cohesive graft condition

### Methods:

1. Evaluated the degree of fluid separation from the graft
   a) “Good”/“Poor”—see above
2. Evaluated the resulting tissue graft condition upon removal from the container
   a) Moldable”—see above
   b) “Cohesive”—see above
3. Evaluated the condition of the graft upon removing from the package
   a) “Saturated”—see above
   b) “Oversaturated”—see above
4. Evaluated Prep time using the packaging=The total amount of time starting from when the user pours fluid used to rehydrate the tissue (step 2b above), until when the tissue is ready to use by the user as noted by appropriate cohesive and moldable properties (step 2e above). Note that the time includes a minimum 2 minute soak time for rehydration.

### Results:

| | Basket and | | Basket and | |
|---|---|---|---|---|
| Clinical Preparation | compression | | Compression | |
| Steps | insert Sample A | | insert Sample B | |
| Add Saline to Container - | 19 | ml | 40-50 | ml |
| Estimated amount of | | | | |
| saline added to | | | | |
| container | | | | |
| Total Time graft | 2 | minutes | 2 | minutes |
| rehydrated in saline | | | | |
| fluid in the packaging | | | | |
| Visible Fluid Separation | Good; | | Good; | |
| from Graft | | | | |
| Graft handling upon | Saturated | | Saturated | |
| removal from packaging | cohesive; moldable | | cohesive; moldable | |
| Step 3. Additional steps | None | | None | |
| to achieve desired graft | | | | |
| handling | | | | |
| Total Estimated | 2-2.5 | minutes | 2-2.5 | minutes |

preparation time

1-2. (canceled)

3. A multi-component packaging system for holding a graft therein during hydrating of the graft, separating excess fluid from a hydrated graft, or both, the packaging system comprising:
a graft holding region for holding the graft and, optionally, a fluid;
a reservoir region for receiving and containing fluid;
a container having one or more walls and a bottom which define the reservoir region, and a top portion having an open top, which allows fluid to be provided to the reservoir region, and an inner top cross-sectional area;
one or more fluid communication means which provides fluid communication between the graft holding region and the reservoir region;
one or more fluid separation features for separating fluid from a graft held in the graft holding region; and
one or more compression features for applying compressive force to a graft while held in the graft holding region and causing displacement of fluid from the graft, from the graft holding region, through the one or more fluid communication means, and into the reservoir region.

4. The multi-component packaging system of claim 3, wherein the graft holding region and reservoir region are at least partially coextensive.

5. The multi-component packaging system of claim 3, wherein the one or more fluid separation features and the one or more compression features are at least partially the same structure or not.

6. The multi-component packaging system of claim 6, wherein a bottom portion of the container has an inner bottom cross-sectional area which is greater than the inner top cross-sectional area and provides an expanded available volume at the bottom portion of the container for receiving and holding fluid separated from a graft.

7. The multi-component packaging system of claim 3, wherein the one or more fluid separation features comprise at least:
a first straining basket with an open top, a bottom, a wall, a first inner cross-sectional area or diameter of uniform size and a first outer cross-sectional area or diameter of uniform size; and
a second straining basket with an open top, a bottom, a wall, a second inner cross-sectional area or diameter of uniform size, and a second outer cross-sectional area or diameter of uniform size,
wherein the first outer cross-sectional area or diameter of the first straining basket is sized and shaped to allow the first straining basket to slidingly fit through the inner top cross-sectional area of the container and into the container, wherein the first straining basket occupies at least a portion of the reservoir region of the container,
wherein the second outer cross-sectional area or diameter of the second straining basket is sized and shaped to allow the second straining basket to slidingly fit through the first inner cross-sectional area of the first straining basket and into the first straining basket, and
wherein the bottom of the first straining basket, the bottom of the second straining basket, and at least a portion of each of the one or more walls of the container, together, define the graft holding region, the graft holding region occupying at least a portion of the reservoir region.

8. The multi-component packaging system of claim 7, wherein, when the second straining basket is slidingly inserted into the first straining basket, the reservoir region includes an upper portion which is within the second straining basket, above the bottom of the second straining basket.

9. The multi-component packaging system of claim 7, wherein the one or more fluid communication means comprise at least a first fluid communication means comprising a mesh material, a plurality of apertures, or both, and is provided either: on at least a portion of the bottom of the first straining basket, or on at least a portion of the bottom of the second straining basket.

10. The multi-component packaging system of claim 7, wherein the one or more fluid communication means comprise at least a first fluid communication means and a second fluid communication means, each of which comprises a mesh material, a plurality of apertures, or both, wherein:
the first straining basket includes the first fluid communication means which is provided on at least a portion of the bottom of the first straining basket, and
the second straining basket includes the second fluid communication means which is provided on at least a portion of the bottom of the second straining basket.

11. The multi-component packaging system of claim 10, wherein the first fluid communication means further comprises: a mesh material forming at least a portion of the wall of the first straining basket, a plurality of apertures provided on at least a portion of the wall of the first straining basket, or a combination thereof.

12. The multi-component packaging system of claim 10, wherein the second fluid communication means further comprises: a mesh material forming at least a portion of the wall of the second straining basket, a plurality of apertures provided on at least a portion of the wall of the second straining basket, or a combination thereof.

13. The multi-component packaging system of claim 10, wherein, the one or more compression features comprise at least a primary compression feature which includes the bottom of the second straining basket,
wherein, when the second straining basket is slidingly inserted into the first straining basket, the bottom of the second straining basket is capable of contacting and applying compressive force against a graft held in the graft holding region, and
when the second straining basket applies compressive force on the graft, fluid is separated and displaced from the graft, and the first communication means and the second communication means allow separated and displaced fluid to leave the graft holding region and enter the reservoir region of the container.

14. The multi-component packaging system of claim 13, wherein, the one or more compression features further comprise a secondary compression feature which includes the bottom of the first straining basket,
wherein, when the second straining basket is slidingly inserted into the first straining basket, the bottom of the first straining basket is capable of contacting and applying compressive force against a side of the graft opposite from the compressive force applied by the bottom of the second straining basket for more efficiently separating and displacing fluid from the graft.

15. The multi-component packaging system of claim 7, wherein one or both of the first straining basket and the second straining basket further includes a vertically extending handle affixed at or near the open top thereof, respectively, which facilitates lifting the second straining basket out of the first straining basket, lifting the first straining basket out of the container, or lifting both the first straining basket and the second straining basket together out of the container.

16. The multi-component packaging system of claim 7, further comprising a lid or cover capable of sealingly closing the open top of the container and retaining the first and second straining baskets and displaced excess fluid in the reservoir region until a user desires to remove the displaced excess fluid.

17. The multicomponent packaging system of claim 3, wherein the container has a uniform inner cross-sectional area which is equal to inner top cross-sectional area and extends from the top portion to the bottom of the container and,
wherein the one or more fluid separation features comprise at least a first plunger which includes:
a first compression plate which is sized and shaped to fit slidingly into the container and extends across the uniform cross-sectional area of the container, and
a first vertically-oriented element which extends upward from the compression plate and facilitates moving the first plunger slidingly in the container and removing the first plunger from the container,
wherein the one or more fluid communication means comprise at least a first fluid communication means which comprises a mesh material, a plurality of apertures, or both, and is provided on at least a portion of the first compression plate.

18. The multicomponent packaging system of claim 17, wherein the first compression plate of the first plunger, the bottom of the container, and at least a portion of each of the one or more walls of the container, together, define the graft holding region, the graft holding region occupying at least a portion of the reservoir region of the container.

19. The multicomponent packaging system of claim 18, wherein the one or more compression features comprise at least the first compression plate of the first plunger and, when the first plunger is slidingly inserted into container, the first compression plate is capable of contacting and applying compressive force against a graft held in the graft holding region, and
when the first compression plate applies compressive force on the graft, fluid is separated and displaced from the graft, and the first communication means allows separated and displaced fluid to leave the graft holding region and enter the reservoir region of the container.

20. The multicomponent packaging system of claim 17, wherein the one or more fluid separation features further comprise a second plunger which includes:
a second compression plate which is sized and shaped to correspond to the first compression plate of the first plunger and which fits slidingly into the container and extends across the uniform cross-sectional area of the container, and
a second vertically-oriented element extending upward from the second compression plate,
wherein the one or more compression features further comprises the second compression plate of the second plunger, and
wherein the first vertically-oriented element of the first plunger includes a vertically-oriented opening therethrough and the second vertically oriented element is positioned on the second compression plate in alignment with the vertically-oriented opening and is sized and shaped to be slidingly received through the vertically-oriented opening when the packaging system is assembled.

21. The multicomponent packaging system of claim 20, wherein, when the packaging system is assembled having the second compression plate of the second plunger slidingly inserted in the container, proximate to or contacting the bottom of the container, and the first compression plate of the first plunger slidingly inserted in the container, above the second compression plate, then the first compression plate, the second compression plate, and at least a portion of each of the one or more walls of the container, together, define the graft holding region, and the graft holding region occupies at least a portion of the reservoir region of the container.

22. The multicomponent packaging system of claim 21, wherein the one or more compression features comprise at least the first compression plate of the first plunger and, when the packaging system is assembled, the first compression plate is capable of contacting and applying compressive force against a graft held in the graft holding region, and
when the first compression plate applies compressive force on the graft, fluid is separated and displaced from the graft, and the first communication means provided on at least a portion of the first compression plate allows separated and displaced fluid to leave the graft holding region and enter the reservoir region of the container.

23. The multicomponent packaging system of claim 22, wherein the one or more fluid communication means further comprise the second compression plate of the second plunger which comprises a mesh material, a plurality of apertures, or both, and is provided on at least a portion of the second compression plate of the second plunger.

24. The multi-component packaging system of claim 22, wherein, the one or more compression features further comprise the second compression plate of the second plunger, and, when the packaging system is assembled, the second compression plate is capable of contacting and applying compressive force against a side of the graft opposite from the compressive force applied by the bottom of the second straining basket for more efficiently separating and displacing fluid from the graft.

25. The multi-component packaging system of claim 20, wherein, when the second vertically-oriented element of the second plunger is assembled with and slidingly received in the vertically-oriented opening of the first vertically-oriented element of the first plunger, the assembled first and second vertically-oriented elements are useful as a handle for concurrently removing the first plunger and second plunger from the container.

26. The multi-component packaging system of claim 20, further comprising a lid or cover capable of sealingly closing the open top of the container and retaining the first and second plungers and displaced excess fluid in the reservoir region until a user desires to remove the displaced excess fluid.

27. A method for separating and displacing fluid from a graft using the multi-component packaging system of claim 3, the method comprising:
assembling the multi-component packaging system with a graft held in the graft holding region,
optionally, adding fluid to the reservoir regions and pausing for a time sufficient to hydrate the graft to a desired degree,
displacing fluid from the graft by applying compressive force to the graft using the one or more compression features,
optionally, driving displaced fluid from the graft holding region, through the fluid communication means, to the reservoir region, using the one or more compression features,
allowing displaced fluid to move from the graft holding region, through the fluid communication means, to the reservoir region, before separating the graft from the reservoir region, and
separating the graft from the reservoir region and displaced fluid therein, if any, using the one or more fluid separation features.
